(19) United States
(12) Patent Application Publication
(10) Pub. No.: US 2006/0134639 A1
(43) Pub. Date: Jun. 22, 2006

(54) Method for the determination of cellular transcriptional regulation

(76) Inventors: Christophe Van Huffel, Namur (BE); Jose Remacle, Malonne (BE); Sven Bulow, Hamburg (DE); Nathalie Zammatteo, Gelbressee (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/818,956

(22) Filed: Apr. 6, 2004

(51) Int. Cl. C12Q 1/68

(52) U.S. Cl. 435/6

(57) ABSTRACT

The present invention relates to a new method for determining the RNAi mediated transcriptional regulation of a cell by the determination of a pattern of at least 3 miRNA detected simultaneously and quantified in the same cell extract. The determination of a pattern of miRNA comprises the steps of: (i) providing an array onto which are fixed capture probes, said capture probes being arranged on pre-determined locations and reflecting the genomic or transcriptional matter of a cell; (ii) isolating a miRNA pool potentially present from a cell; (iii) elongating or ligating said miRNAs into labeled capture probes, (iv) contacting said labeled polynucleotides with the array under conditions allowing hybridization of the labeled polynucleotides to complementary capture probes present on the array; (v) detecting and quantifying a signal present on the specific locations of the array, wherein the detection of a pattern of at least 3 signals on the array reflects the pattern of miRNAs being involved in the RNAi mediated cellular transcriptional regulation.

### FIELD OF THE INVENTION

The present invention relates to the field of interference RNA (RNAi). In particular, the present invention relates to a method for the determination of the cellular transcriptional regulation based on a simultaneous detection and quantification of a pattern of miRNAs in a cell.

### DESCRIPTION OF THE RELATED ART

In experiments, during which dsRNA was injected into the nematode Caenorhabditis elegans it was found that a silencing of genes highly homologous in sequence to the delivered dsRNA occurred (Fire et al., 1998 Nature 391:806-811). Based on this finding the term “RNA interference” (RNAi) was created nominating the capability of such dsRNA-molecules to affect the translation of transcripts.

During ensuing research in this area it has been shown that dsRNA triggers degradation of homologous RNAs within the region of identity with the dsRNA (Zamore et al., 2000 Cell 101:25-33). Apparently, the dsRNA is processed to RNA fragments exhibiting a length of about 21-23-ribonucleotides (Zamore et al., 2000 Cell 101:25-33). These short fragments were also detected in extracts prepared from Drosophila melanogaster Schneider 2 cells that were transfected with dsRNA before cell lysis (Hammond et al., 2000 Nature 404:293-296) or after injection of radiolabeled dsRNA into D. melanogaster embryos (Yang et al.,2000 Curr. Biol. 10:1191-1200) or C. elegans adults (Parrish et al., 2000 Mol. Cell 6:1077-1087).

RNAi was observed to also be naturally present in a wide range of living cells. E.g. these kind of molecules has been found to exist in insects (Kennerdell and Carthew, 1998 Cell 95:1017-1026), frog (Oelgeschlager et al. 2000 Nature 405:757-763), and other animals including mice (Svoboda et al., 2000 Development 127:4147-4156; Wianny and Zernicka-Goetz, 2000 Nat. Cell Biol. 2:70-75) and also in humans. RNA molecules of similar size have also been found to accumulate in plant tissue that exhibits post-transcriptional gene-silencing (PTGS) (Hamilton and Baulcombe, 1999 Sciences 286:950-952).

The natural function of RNAi and co-suppression appears to be the protection of the genome against invasion by mobile genetic elements, such as transposons and viruses, which produce aberrant RNA or dsRNA in the host cell when they become active (Jensen et al., 1999 Nat. Genet. 21:209-212; Ketting et al.,1999 Cell 99:133-141; Ratcliff et al., 1999 Plant Cell 11:1207-1216; Tabara et al., 1999 Cell 99:123-132; Malinsky et al., 2000 Genetics 156:1147-1155). Specific mRNA degradation prevents transposon and virus replication, although some viruses seem to be able to overcome or prevent this process by expressing proteins that suppress PTGS (Anandalakshmi et al., 2000 Science 290:142-144; Lucy et al., 2000 EMBO J. 19:1672-1680; Voinnet et al., 2000 Cell 103:153-167).

The currently existing model for the mechanism of RNAi is based on the observation that the introduced dsRNA is bound and cleaved by RNase III-like enzyme Dicer to generate products having the length indicated above. These molecules, termed small interfering RNAs (siRNAs) trigger the formation of RNA-induced silencing complex (RISC). The resulting dsRNA-protein complexes appear to represent the active effectors of selective degradation of homologous mRNA (Hamilton and Baulcombe, 1999 Science 286:950-952, Zamore et al., 2000 Cell 101:25-33; Elbashir et al., 2001. Genes & Dev. 15:188-200). Elbashir et al. provide evidence that the direction of dsRNA processing determines, whether sense or antisense target RNA can be cleaved by the siRNA-protein complex. Helicases in the complex unwind the dsRNA, and the resulting single-stranded RNA (ssRNA) seems to be used as a guide for substrate selection. Once the ssRNA is base-paired with the target mRNA, a nuclease activity, presumably within the complex, degrades the mRNA.

The enzymatic machinery for generating siRNA also appears to be used for the production of a second class of endogenously encoded, small RNA molecules termed microRNAs (miRNAs). miRNAs regulate mRNA translation whereas siRNAs direct RNA destruction via the RNA interference pathway. miRNAs are processed through at least two sequential steps; generation of 70 nucleotides (pre-miRNAs) from the longer transcripts (termed pri-miRNAs) and processing of pre-miRNAs into mature miRNAs (Lee et al. 2002 EMBO J. 21:4663-4670). miRNAs are typically 20-22 nucleotides non-coding RNA that regulate expression of mRNA exhibiting sequences complementary thereto. They are numerous and widespread among eukaryotes, being conserved throughout evolution.

Research on miRNAs is increasing as scientists are beginning to appreciate the broad role that these molecules play in the regulation of eukaryotic gene expression. There is speculation that miRNAs may represent a newly discovered layer of gene regulation. As a result, there is intense interest among researchers around the world in the targets and mechanism of action of miRNAs.

The two best understood miRNAs, lin-4 and let-7, regulate developmental timing in C. elegans by regulating the translation of a family of key mRNAs (Pasquinelli and Ruvkun 2002 G Ann Rev Cell Dev Biol. 18:495-513). Several hundred miRNAs have been identified in C. elegans, Drosophila, mouse, and humans. As would be expected for molecules that regulate gene expression, miRNA levels have been shown to vary between tissues and developmental states.

In human, there are between 200 and 300 miRNA genes and about 200 have been identified at the moment. In heart, liver or brain, it is found that a single, tissue-specifically expressed miRNA dominates the population of expressed miRNAs and suggests a role for these miRNAs in tissue specification or cell lineage decisions (Lagos-Quintana et al. 2002 Current Biology 12:735-739).

Characterization of a number of miRNAs indicates that they influence a variety of processes, including early development (Reinhart et al. 2000 Nature 403:901-906), cell proliferation and cell death (Brennecke et al. 2003 Cell 113:25-36), and apoptosis and fat metabolism (Xu et al. 2003 Curr. Biol. 13:790-795). In addition, one study shows a strong correlation between reduced expression of two miRNAs and chronic lymphocytic leukemia, providing a possible link between miRNAs and cancer (Calin et al., 2002 PNAS USA 99:15524-15529). Although the field is still young, there is speculation that miRNAs could be as important as transcription factors in regulating gene expression in higher eukaryotes.

miRNAs affects the expression of target genes by one of at least two mechanisms. Some bind to the 3′UTR of target mRNAs and suppress translation (Chi et al., 2003 PNAS USA 100:6343-6346). Others act as siRNAs, binding to and destroying target transcripts. miRNAs interfere with expression of messenger RNAs encoding factors that control developmental timing, stem cell maintenance, and other developmental and physiological processes in plants and animals. miRNAs are negative regulators that function as specific determinants, or guides, within complexes that inhibit protein synthesis (animals) or promote degradation (plants) of mRNA targets (Carrington and Ambros, 2003 Science 301:336-338). Plants with altered miRNA metabolism have pleiotropic developmental defects. In Arabidopsis, a miRNA has been identified “JAW” that can guide messenger RNA cleavage of several TCP genes controlling leaf development (Palatnik et al., 2003 Nature 425:257-263).

Recently, miRNAs have been identified in undifferentiated and differentiated mouse embryonic stem (ES) cells (Houbaviy et al. 2003 Dev Cell 5:351-358). Their expression is repressed as ES cells differentiate into embryoid bodies and is undetectable in adult mouse organs. In contrast, the levels of many previously described miRNAs remain constant or increase upon differentiation. These results suggest that miRNAs may have a role in the maintenance of a pluripotent cell state and in the regulation of early mammalian development.

Finally, miRNA mechanism of action is diverse and does not only target RNA transcript. miRNA's may also regulate gene expression by causing chromatin condensation. Several groups have shown that binding of dsRNAs to plant-promoter regions can cause gene silencing—an effect that is mediated via DNA methylation.

The detection of miRNA is difficult to perform given their diversity, their small size and their low copy number in the cells. Because their small size precludes the use of RT-PCR, most miRNA researches currently use Northern blot analysis with polyacrylamide gels and radioactive labeling to examine miRNA expression. This technique is labor intensive, requires large amounts of RNA and is restricted to the analysis of one miRNA at a time. Moreover, the use of radiolabeled probes is not recommended for routine tests because of their short half-life and the requirement of safe infrastructure and waste release.

Transcriptional regulation of multiple gene expression is a complex and subtle process. In order to investigate the effect of the miRNA on their transcribed genes, the assay has to be quantitative since small variation in their amount affects the gene expression in a significant way and modifies the cell composition.

Thus, there is a need in the art for a sensitive method to determine, whether a cell is subject to an RNAi mediated transcriptional regulation.

### SUMMARY OF THE INVENTION

It is, therefore, an object of the present invention to provide a method for rapidly and reliably detecting and quantifying the cellular transcriptional regulation mediated by RNAi.

In accomplishing these and other objects of the invention, there is provided, in accordance with one aspect of the invention, a method for determining the RNAi mediated transcriptional regulation of a cell by the determination of a pattern of at least 3 miRNA detected simultaneously and quantified in the same cell extract. The method of determination of a pattern of miRNA comprises the steps of: (i) providing an array onto which are fixed capture probes, said capture probes being arranged on pre-determined locations thereon and reflect the genomic or transcriptional matter of a cell, (ii) isolating miRNAs potentially present from a cell, (iii) elongating or ligating said miRNAs into target labeled polynucleotides, (iv) contacting said target labeled polynucleotides with the array under conditions allowing hybridization of the target labeled polynucleotides to complementary capture probes present on the array, (v) detecting and quantifying a signal present on a specific locations of the array, wherein the detection of a pattern of at least 3 signals on the array reflects the pattern of miRNA being involved in the RNAi mediated cellular transcriptional regulation.

The method according to the present invention may further comprise the step of correlating the cell transcriptional regulation provided by the detection and quantification of a pattern of miRNAs with the pattern of expression of the regulated genes in the same sample.

The presence and the amount of the different miRNA may be correlated with the amount of the different genes for which transcription is under the control of these different miRNA.

In one embodiment, the detected miRNAs are mature miRNAs.

In one embodiment, the cellular transcriptional regulation provided by the detection and quantification of a pattern of is correlated with the pattern of expression of the miRNA targeted genes in the same sample. In another embodiment, the cell transcriptional regulation provided by the detection and quantification of a pattern of miRNAs is correlated with the pattern of expression of the genes having mRNA sequences complementary to the corresponding miRNA sequences detected in the same sample.

In another embodiment, the invention provides a method, wherein the cellular transcriptional regulation is related to one the following fields: development, cell differentiation or stem cell maintenance, cell proliferation, cell death, chromatin condensation or cell transformation.

In still another embodiment, the detection of the miRNA is performed after elongation of the miRNA hybridized on its complementary bait sequence with the Tth DNA polymerase 3. In another embodiment, the AMV or the M-MLV reverse transcriptase may be used for carrying out the elongation reaction.

In an alternative embodiment, the DNA/DNA-RNA hybrid complex obtained by elongation is then amplified by any linear amplification methods such as in vitro RNA transcription, asymmetric or linear PCR. In a preferred embodiment, one primer is provided for linear amplification of the elongated sequences. Quantification of the multiple miRNA present in a sample is provided by one simple treatment of all the miRNA and direct hybridization on their corresponding capture

In another embodiment, the detection of the miRNA is performed after ligation of the miRNA hybridized on its complementary bait sequence with an adjacent probe. In another embodiment, the adjacent probe is pre-hybridized with its complementary bait sequence before ligation with the miRNA. In still another embodiment, the T4 RNA ligase may be used for carrying out the ligation reaction. In a preferred embodiment, the adjacent probe is labeled.

The invention further provides kits for the determination of cellular transcriptional regulation in a sample comprising an array comprising capture probes being arranged on pre-determined locations and having sequences identical or complementary to miRNAs of interest or parts thereof and optionally, buffers and labels. In another embodiment, the kit may also comprise a second array for the detection and quantification of the expression of the regulated genes in the same sample.

Other objects, features and advantages of the present invention will become apparent from the following detailed description. The detailed description and specific example, while indicating preferred embodiments, are given for illustrative purposes only, since various changes and modifications within the spirit and scope of the invention will become apparent to those skilled in the art from this detailed description. Further, the example demonstrates the principle of the invention and cannot be expected to specifically illustrate the application of this invention to all the examples where it will be obviously useful to those skilled in the prior art.

### BRIEF DESCRIPTION OF THE DRAWINGS

FIG. 1 shows an embodiment for the detection of miRNAs on arrays. The miRNAs are first incubated in solution with a mixture of single strand DNA baits having part of their sequence complementary to the miRNAs. After elongation and labeling, they are detected by hybridization on array bearing sequences identical at least partly to the miRNA for which the analysis is required.

FIG. 2 shows an alternative embodiment of FIG. 1. The single strand DNA baits are composed of three parts: the 3′ end is complementary of the miRNA, the middle part is specific of each bait and the 5′ end sequence is common to all baits. After hybridization of the miRNA and their elongation, the miRNA is degraded. A primer complementary of the common sequence of the elongated DNA is provided for linear amplification. Labeling occurs during the amplification by the incorporation of labeled nucleotides by a DNA polymerase. After linear amplification, the labeled products are detected on an array bearing sequences complementary at least partly to the amplified product.

FIG. 3 shows an embodiment for the miRNAs detection in which the miRNAs are hybridized with a mixture of single strand DNA baits which are pre-hybridized with labeled probes being adjacent to the miRNAs possibly present in the sample. After ligation of the miRNAs with the adjacent probes, the ligated products are detected on an array bearing sequences identical at least partly to the miRNA for which analysis is required.

### DETAILED DESCRIPTION OF THE PREFERRED EMBODIMENT

Definitions

The term “genes” shall designate the genomic DNA which is transcribed into RNA and then optionally translated into peptides or proteins. The measurement of the expressed genes is performed on either molecules within this process most currently the detection of the mRNA or of the peptide or protein. The detection can also be based on specific property of the protein being for example its enzymatic activity.

The terms “nucleic acid, array, probe, target nucleic acid, bind substantially, hybridizing specifically to, background, quantifying” are as described in the international patent application WO97/27317, which is incorporated herein by reference.

The term “nucleotide triphosphate” refers to nucleotides present in either as DNA or RNA and thus includes nucleotides which incorporate adenine, cytosine, guanine, thymine and uracil as bases, the sugar moieties being deoxyribose or ribose. Other modified bases capable of base pairing with one of the conventional bases adenine, cytosine, guanine, thymine and uracil may be employed. Such modified bases include for example 8-azaguanine and hypoxanthine.

The term “nucleotide” as used herein refers to nucleotides present in nucleic acids (either DNA or RNA) compared with the bases of said nucleic acid, and includes nucleotides comprising usual or modified bases as above described.

References to nucleotide(s), oligonucleotide(s), polynucleotide(s) and the like include analogous species wherein the sugar-phosphate backbone is modified and/or replaced, provided that its hybridization properties are not destroyed. By way of example, the backbone may be replaced by an equivalent synthetic peptide, called Peptide Nucleic Acid (PNA).

The terms “nucleotide species” is a composition of related nucleotides for the detection of a given sequence by base pairing hybridization; nucleotides are synthesized either chemically or enzymatically but the synthesis is not always perfect and the main sequence is contaminated by other related sequences like shorter one or sequences differing by a one or a few nucleotides. The essential characteristic of one nucleotides species for the invention being that the overall species can be used for capture of a given sequence.

“Polynucleotide” sequences that are complementary to one or more of the miRNA described herein, refer to capture probes that are capable of hybridizing under stringent conditions to at least part of the nucleotide sequence of said miRNA or miRNA copies. Given the small size of the miRNA, the capture molecules have to be identical or at least have more than 90% identical sequence in order to specifically detect the miRNA beside other possible flanking regions such as spacer sequences.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “capture probe” designates a molecule which is able to specifically bind to a given polynucleotide. Polynucleotide binding is obtained through base pairing between two polynucleotides, one being the immobilized capture probe and the other one the target to be detected.

The term, the genomic or transcriptional matter of a cell shall designate the genes and/or the genome and/or the transcripts represented by RNA in the cell.

The present invention is based on the use of arrays having multiple single nucleotide sequences arranged on specific, pre-determined locations thereon and being identical or complementary to miRNA, present in the cells for which the pattern of transcriptional regulation is to be determined.

The main characteristic of the invention is to obtain a pattern of transcriptional regulation based on the simultaneous detection and quantification of multiple miRNAs present in a cell. The signals of the different spots related to each gene being a direct measurement of the diversity and the concentration of the miRNA in the analysed cells or tissues. Also, the invention is not limited by the number of miRNA to be screened. The array allows to analyse either from 5 to 500 and more preferably until 5000 miRNAs in a cell. This number depends on the species and the number of expressed miRNA genes in the analysed cells.

The present invention provides a method for the determination of cellular transcriptional regulation by the simultaneous detection and quantification of multiple miRNAs present in a cell on an array and by detecting a signal present on a specific location on the array, said signal at such location being related to the presence of one miRNA with the detection of at least 3, preferably at least 5, more preferably at least 10 and even more preferably at least 20 miRNAs on the array being indicative of a given miRNA or RNAi mediated cellular transcriptional regulation.

In general, in a cell, there are typically about 20 miRNA genes expressed. In human there are about 200 to 300 miRNA genes. The identification of a pattern of expressed miRNAs in a given cell brings an answer to the question, whether a cell is subject to RNAi mediated transcriptional regulation (e.g. the genes regulated by these miRNA and their target genes). In a preferred embodiment, to unravel the cellular transcriptional regulation, the pattern of at least 3 miRNAs obtained by the method of the invention is correlated with the pattern of expression of the regulated genes in the same sample (e.g. provided by a second array). In another embodiment, the pattern of at least 3 miRNA is correlated with the pattern of expression of the miRNA target genes in the same sample (e.g. provided by a second array). In an alternative embodiment, the pattern of at least 3 miRNA is correlated with the pattern of expression of genes having miRNA sequences complementary to the corresponding miRNA sequence in the same sample (e.g. provided by a second array). In another embodiment, the pattern of at least 3 miRNAs obtained by the method of the invention is correlated with activated transcriptional factors in the same sample.

In a preferred embodiment, the invention provides a method for the simultaneous detection of at least 3 of the miRNA presented in Table 1 for human cells and at least 3 miRNA presented in Table 2 for mouse cells.

In a preferred embodiment, the invention provides a method wherein the cellular transcriptional regulation is related to one the following fields: development, cell differentiation or stem cell maintenance, cell proliferation, cell death, chromatin condensation or cell transformation.

In principle, the method comprises the steps (step (i)) of providing a support containing an array onto which a number of capture probes are arranged on pre-determined locations. These capture probes relate to miRNA sequences or their complement and/or genes and/or transcripts of a cell.

The support is generically composed of a solid surface which may be selected from the group consisting of glasses, electronic devices, silicon supports, silica, metal or mixtures thereof prepared in format selected from the group of slides, discs, gel layers and/or beads. Beads are considered as arrays in the context of the present invention, as long as they have characteristics which allow a differentiation from each other, so that identification of the beads is correlated with the identification of a given capture probe and so of the target sequence.

On the support, a number of capture molecules is fixed by covalent binding, each capture molecule being located at a specific location and having at least in part a sequence in a single strand form identical to a miRNA or its complement for which the presence is screened.

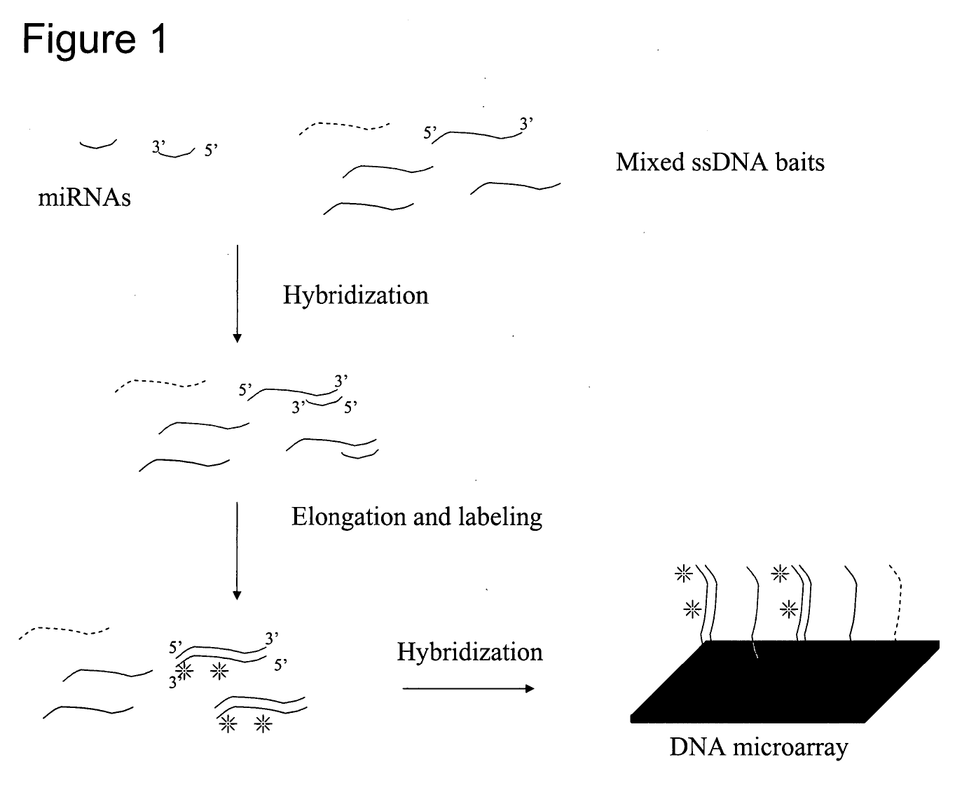

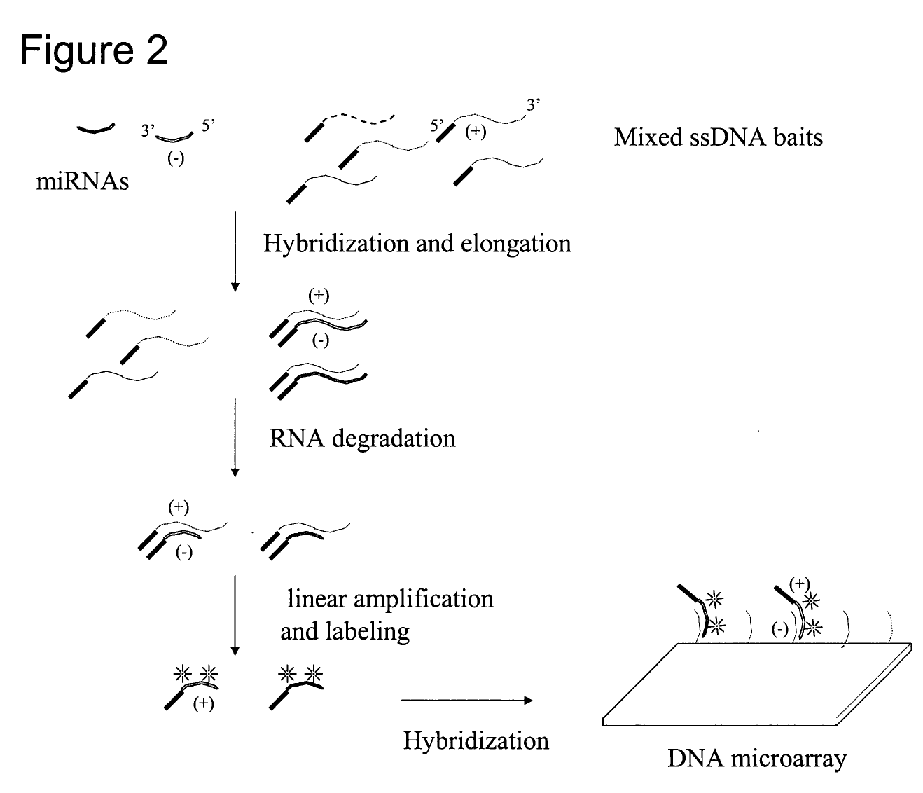

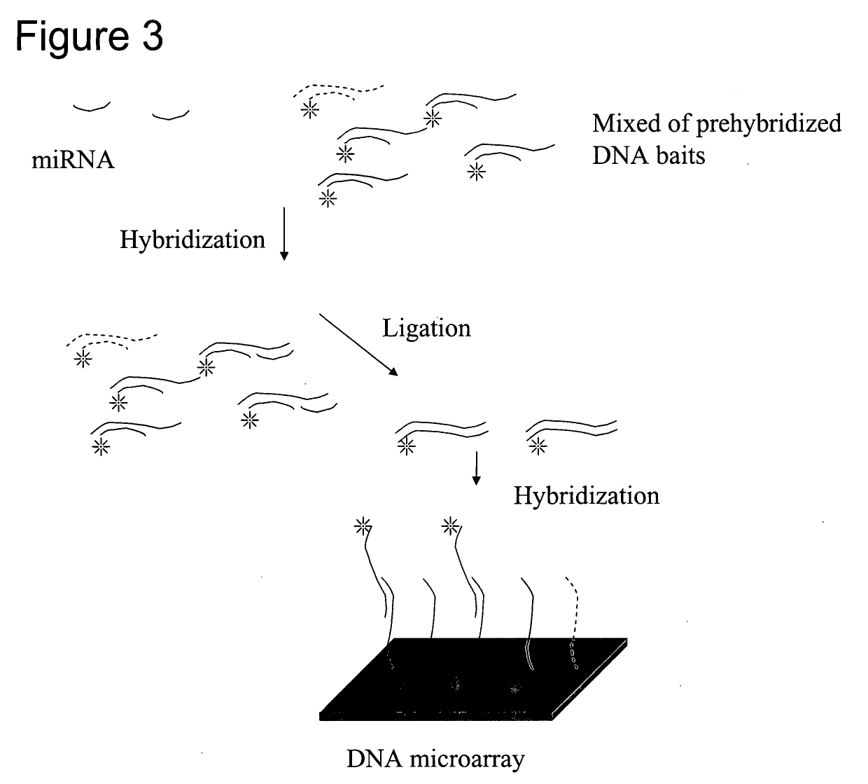

In principle, the detection of the miRNAs may be performed on the same strand of the corresponding miRNA sequence (−) or on its complementary sequence (+), since the miRNAs contain one strand (−). However, in some applications, the miRNAs may be complementary to mRNA (+) and the use for the detection of strand identical to the miRNA sequences (−) might lead to a binding of the natural mRNA (+) as well, which might interfere with the analysis. This is the case for the detection method presented in FIGS. 1 and 3. In this case, the sequence (+) complementary to the miRNA (−) has to be present on the surface of the array. In the method proposed in FIG. 2, there is not interference with mRNA since the detected sequences have the identical strand.

Generally, the capture probes may be synthesized by a variety of different techniques, but are preferably synthesized by PCR amplification from cloned genes using an aminated primer. The amino group of the amplicon is then reacted with a functionalized surface bearing reactive groups, such as, but not limited, to aldehyde, epoxide, acrylate, thiocyanate, N-hydroxysuccinimide. After having formed a covalent linkage, the second strand of the amplicon is then removed by heating or by alkaline treatment so that single strand DNA or RNA is present on the surface and ready to bind to the miRNA or its complement.

Given the progress of chemical synthesis of the nucleotides, the use of chemically synthesized nucleotides is also envisaged in the invention. The synthesized nucleotides are also preferably aminated or thiolated and deposited on the functionalized surface. Advantage of the chemically synthesized nucleotides is their easy production but the limitation is the yield of each of the successive addition of nucleotides making the capture probes not unique especially when long polynucleotide capture probes are synthesized. We will then considered a chemically synthesized capture probe typically as a capture probe species.

In a preferred embodiment the array comprises capture probes represented by polynucleotides having a sequence identical to miRNA. In another embodiment, the array contains capture probes being polynucleotides having a sequence complementary to miRNA.

Preferably, the different capture molecules present on the array cover most and preferably all of the miRNA present in a cell. In an embodiment, the array comprises 5-200, preferably 5-500, 5-1000 and even 5-5000 capture probes specific of the miRNA or their complement.

In principle, the micro-array may contain at least 3 capture probes, e.g. one capture probe associated with each miRNA. Yet, the number of capture probes on the micro-array may be selected according to the need of the skilled person and may contain capture probes for the detection of up to about 5000 different miRNA, e.g. about 100, or 200 or 500 or 1000, or 2000 different miRNA involved in the cell transcriptional regulation.

The array may target human or mouse miRNA sequences as listed in Table 1 and Table 2 respectively. Similar array can be constructed for other species including rat, Drosophila, Arabidopsis and Caenorhabditis with some adaptation in the sequence for the capture probes.

According to a preferred embodiment the capture probes are polynucleotides and are unique for each miRNA to be detected and quantified on the array. The minimum length corresponds to the size of the miRNA or their complement. The capture probes may comprise a spacer sequence which is not related to the miRNA sequences and which do not react significantly with these sequences. Spacer to be used in the present invention may be obtained following methods described in the WO0177372, which document is incorporated herein by way of reference. The overall length of the capture probes (including the spacer) may range from about 15 to about 1000 nucleotides, preferably of from about 15 to about 400, or 15 to 200 nucleotides, or more preferred of from about 15 to about 100, or from 15 to 50, or from 15 to 30 and are fixed on a support being any solid support as long as they are able to hybridize with their corresponding miRNA or their complement and be identified and quantified.

In a preferred embodiment, the capture probes have sequences which are identical for at least 10 to 1000 nucleotides to the same part of the mRNA corresponding to the miRNA to be detected. In another embodiment, an individual capture probe is used for the detection of several miRNAs directed to the same mRNA.

In a preferred embodiment, the capture probes are chosen in order to obtain a quantitative hybridization of the target nucleotides meaning a reproducible detection with a coefficient of variation below 30% and preferably below 20% for the same target nucleotide in separated experiments and below 50% and preferably below 25% for different target nucleotides present in the same solution.

In another preferred embodiment, the capture molecules are present at a density superior to 10 fmoles, and preferably 100 fmoles per cm² surface of the solid support. In another embodiment capture probes are present on different supports being preferentially beads with chemical or physical characteristics for their identification with a specific capture probe.

The invention also embraces the support and its substrate on which is bound the capture molecules for the detection of given mRNA target molecules which are complementary to the corresponding miRNA sequences.

Methods of arranging nucleotides and polynucleotides on an array are well known in the art and may be found in Bowtell, D. and Sambrook (DNA Microarrays, J. Cold Spring Harbor Laboratory Press, 2003 Cold Spring Harbor, N.Y., 1-712) which document is incorporated herein by way of reference. In a preferred embodiment the nucleotide sequence is attached to the support via a linker, which may be a polynucleotide exhibiting a length of between about 20 to 200 nucleotides (WO0177372). In principle, the capture probes may be DNA, PNA or RNA.

In a next step (step (ii)), miRNA from a cell of interest is isolated. An exemplary process for the isolation of small interference RNA (siRNA) is described e.g. in Tuschl et al. 1999 Genes & Dev. 13:3191-3197, which document is incorporated herein by way of reference.

The miRNA once isolated will be labeled prior to its use (step (iii)). The miRNA is incorporated into a labeled DNA-RNA sequence which is then detected on the array. The labeling may be performed by elongating or ligating the miRNA. In a preferred embodiment, the labeling may be performed by incubating the miRNA with a mixture of ssDNA under conditions as to obtain formation of a RNA-DNA hybrid complex, whereupon an elongation and concurrent labeling of the small miRNA may be achieved. Here, the ssDNA bait is used as a matrix and labeled ribonucleotides/deoxynucleotides are utilized for the elongation of miRNA. The ssDNA bait used for the formation of the hybrid complex can be replaced by any nucleotide or nucleotide-like molecules as long as the elongation of the bound miRNA is possible. After denaturation, the labeled strand will be used for incubation with the capture probes present on the array for detection and quantification of the miRNA (FIG. 1).

According to a preferred embodiment, the elongation is performed with the Tth DNA polymerase 3 which accept as primer RNA sequences such as miRNAs. The elongated and labeled polynucleotide is DNA. In another embodiment, the elongation is obtained with the AMV or with the M-MLV reverse transcriptase.

In a preferred embodiment, the ssDNA bait is a sequence identical to the mRNA strand or part of it (+) which is complementary to the corresponding miRNA (−) for which the analysis is required. After elongation and labeling, the elongated strand (−) is hybridized on a capture probe (+) identical to the mRNA strand or part of it. The same capture probe may be used for parallel detection and quantification of the mRNA present in the same sample. After retro-transcription of the mRNA (+), the labeled cDNA (−) is hybridized on the same capture probe. This method greatly simplifies the production of the capture probes which are equivalent for both applications.

In a preferred embodiment, the mixture of ssDNA baits is composed of three parts: the 3′ end is complementary of the miRNA, the middle part is specific of each bait and the 5′ end sequence is common to all baits. After hybridization of the miRNA and their elongation, the product is amplified. After degradation of the miRNA, the matrix for the amplification is a DNA/DNA hybrid complex. A primer complementary of the common sequence of the elongated DNA is provided for linear amplification with a DNA polymerase. Advantageously, only one primer is required for the amplification of all elongated miRNAs. Altered cycles of denaturation, primer annealing and polymerization are performed like in a normal PCR except that only one primer is used which results in a linear amplification. The advantage of such amplification is that quantification of the initial amount of miRNA remains possible due to the linearity of the amplification. After linear amplification, the products are detected on an array bearing sequences complementary at least partly to the amplified product (FIG. 2). The target labeled nucleotides which are hybridized on the array are preferably labeled during the amplification. Preferably, the capture probes of the array do not comprise the primer sequence used for the amplification nor the miRNA sequences or their complement. In order to avoid interference between the ssDNA baits (+) introduced at the beginning of the assay with the amplified labeled products (+) for the hybridization on the capture probes (−) of the array, the mixture of ssDNA baits may be specifically degraded before the amplification (e.g. by S1 nuclease).

Alternatively, the primer complementary of the common sequence of the elongated DNA may comprise a T7 promoter sequence for an RNA polymerase that might be used for in vitro transcription. The primer may also comprise a Tag sequence which is used for further amplification (e.g. the tag may be a sequence rich in cytosine if the amplification is performed with labeled CTP, thus increasing the number of incorporated label during the amplification).

The labeling may be obtained by the incorporation of labeled ribonucleotides/deoxynucleotides during the amplification step in order to obtain target labeled polynucleotides according to the invention. Fluorescent labeled nucleotides are preferred since they are incorporated by the polymerase and lead to the formation of fluorescent labeled target polynucleotides. Cy3, Cy5 or Cy7 labeled nucleotides are preferred fluorochromes.

In another embodiment, the detection of the miRNA is performed after ligation of the miRNA hybridized on its complementary bait sequence with an adjacent probe.

The labeling may be performed by ligating the miRNA. In a preferred embodiment, the labeling of the miRNA is performed after ligation of the miRNA hybridized on its complementary bait sequence with an adjacent probe. Labeling may be obtained by using a labeled adjacent probe for ligation. In a preferred embodiment, the adjacent probe is pre-hybridized with its complementary bait sequence before ligation with the miRNA. Ligation may be performed under conditions as to obtain formation of a DNA/DNA-RNA hybrid complex. Here, the DNA bait is used as a matrix and the DNA adjacent probe is utilized for ligation with miRNA. In a preferred embodiment, the ssDNA bait is a sequence identical to the mRNA strand or part of it which is complementary to the corresponding miRNA for which the analysis is required. The DNA baits used for the formation of the hybrid complex can be replaced by any nucleotide or nucleotide-like molecules as long as the ligation of the bound miRNA is possible. After denaturation, the labeled strand will be used for incubation with the capture probes present on the array for detection and quantification of the miRNA (FIG. 3). Preferably, the capture probes of the array are not complementary of the labeled adjacent probes in order to avoid false positive hybridizations.

According to a preferred embodiment, the ligation is performed with the T4 RNA ligase which accept to ligate DNA sequences to RNA sequences such as miRNAs.

Preferred labels may be detected, e.g. via fluorescence, colorimetry, chemo- or bioluminescence, electric, magnetic or particularly biotin. Biotin-labeled nucleotides may be attached/incorporated, which is then recognized by binding proteins being either antibodies or streptavidin or related binding molecules. The binding proteins are labeled by any chemical or physical means and detected and quantified. Indirect labeling is also of use when amplification of the signal is required.

In a next step (step (iv)), the labeled miRNA or molecule derived therefrom (e.g. a DNA-copy or amplified RNA), is contacted with the array under conditions, allowing hybridization of the labeled miRNA, or the molecule derived therefrom, with the capture probes present on the array. After a time sufficient for forming the duplex, a signal is detected on a specific location on the array (step (v)). The detection of a pattern of at least 3 signals on the array reflects the pattern of miRNA being involved in the RNAi mediated cellular transcriptional regulation.

In a preferred embodiment, the signal present on a specific location on the array corresponds to a pattern of at least 5, 10, 15, 20, 25, 30 and even 50 miRNAs.

In case the miRNA, or molecule derived therefrom, has been labeled prior to the hybridization step, the presence of fixed labeled target will be indicative of the presence of miRNA in the sample. The amount of fixed labeled target on the array will be proportional to the miRNA if performed under the appropriate conditions.

The presence of target bound on the different capture probes present on the solid support may be analyzed, identified and quantified by an apparatus comprising a detection and quantification device of a signal formed at the location of the binding between the target molecule and the capture molecule, preferably also a reading device of information recorded on a surface of said solid support, a computer program for recognizing the discrete regions bearing the bound target molecules upon its corresponding capture molecules and their locations, preferably also a quantification program of the signal present at the locations and a program for correlating the presence of the signal at these locations with the diagnostic and the quantification of the components to be detected according to the invention.

Therefore, the method as described herein may be utilized as part of a diagnostic and quantification kit which comprises means and media for performing an analysis of biological samples containing target molecules being detected after their binding to the capture probes being present on the support in the form of array with a density of at least 5 different capture probes per cm² of surface of rigid support.

Also provided by the present invention is a kit for the determination of cell transcriptional regulation in a sample, which kit comprises an array, harboring capture probes having a sequence identical or complementary to a miRNA or parts thereof and being present at pre-determined locations of the array, and buffers and labels. In another embodiment, the kit may also comprise a second array for the detection and quantification of the expression of the regulated genes in the same sample. The aim of this second array is to obtain a direct analysis and an overview of the genes which are essentially affected by the regulation through miRNAs present in the cells. In another embodiment, the detection and quantification of the expression of the regulated genes is obtained through the detection and quantification of mRNA or proteins in the same sample. In a preferred embodiment, the two arrays are present on the same support and allow a direct comparison between the pattern of miRNAs present in the sample with the pattern of genes differentially expressed in the same sample. In another embodiment, the two arrays are present on different supports. In another embodiment, the capture probes of the arrays for the detection of the miRNAs and of the mRNA are nucleotide sequences having part of their sequence identical to the mRNA. The advantage of such system is that the same capture probes present on the same support may be used for the detection of both the miRNA and their corresponding mRNA.

The signals of the different spots related to each gene are a direct measurement of the diversity and the concentration of the expressed genes (mRNA) in the analysed cells or tissues. The amount of genes is directly related to the presence and amount of the corresponding miRNA and their regulating activity of the corresponding genes. In this embodiment, the results obtained on the gene expression array is directly related to the results obtained on the miRNA array and conclusions may be drawn on the transcriptional regulation of these individual genes in the particular analysed sample. In particular, the variation in the amount of one miRNA in a test sample compared to a reference sample may be correlated with the change in the amount of transcribed mRNA corresponding gene. This correlation may be performed for at least 3 miRNA and a pattern of transcriptional regulation provided by the presence of the miRNA will be constructed. Such regulation may than be correlated to the biological situation from which the sample was derived for the analysis, being a biological or pathological or an experimental set up.

The invention is not limited by the number of genes to be screened. The array allows to analyse either from 2 to 100 and more preferably until 1000 and still up the entire gene pool present in a cell. This number depends on the species but can be as large as around 40.000 for the human genome.

The following example illustrate the invention without limiting it thereto.

### EXAMPLE

The experiment is performed as schematically described in FIG. 1.

miRNA extraction:

miRNAs are extracted from a fresh (or frozen) pellet of 10² to 10⁷ cultured cell or tissues using the mirVana miRNA isolation procedure variant for isolation of RNA that is highly enriched for small RNAs (Ambion). The sample was disrupted in a denaturing lysis buffer and subsequently extracted in Acid-Phenol:Chloroform (Chomczynski and Sacchi, 1987 Anal. Biochem. 162:156-159) ⅓ volume of 100% ethanol is added to the aqueous phase recovered from the organic extraction, mixed and passed through a glass filter cartridge (using centrifugal force). After this step, the filtrate was further enriched by adding ⅔ volume of 100% ethanol, mixed and applied on a second glass filter cartridge. The small RNA molecules remain trapped on the glass filter and are washed three times with a 45% ethanol solution. The RNA is then eluted with nuclease-free water and recovered in a collection tube.

miRNA Labelling:

The small size RNA population is then annealed, with a mixture of single stranded DNA molecules by heating at 95° C. for 5 min and annealing for 30 min at 37° C. The annealed RNA-DNA hybrids are then labeled with biotin labeled dNTPs using Tth DNA polymerase 3 holoenzyme. miRNA strand extension is accomplished by incubation of the RNA-DNA mixture with Tth DNA polymerase 3 holoenzyme mix for 20 s. in a buffer containing 20 mM Taps-Tris pH 7.5, 8 mM Mg(OAc)₂, 0.04 mg/ml BSA, 40 μM Sorbitol, 0.5 mM ATP and 0.2 mM labeled dNTP mix). The reaction is quenched by adding EDTA at a final concentration of 10 mM, vortexing and is maintained on ice.

Hybridization

The resulting product is then hybridized on the DualChips miRNA micro-array bearing ssDNA capture probes specific for miRNA sequences (Eppendorf, Hamburg, Germany).

Hybridization chambers were from Biozym (Landgraaf, The Netherlands). Hybridization mixture consisted in biotinylated miRNA-DNA hybrid, 6.5 μl HybriBuffer A (Eppendorf, Hambourg, Germany), 26 μl HybriBuffer B (Eppendorf, Hambourg, Germany), 8 μl H₂O, and 2 μl of positive hybridization control.

Hybridization was carried out overnight at 60° C. The micro-arrays were then washed 4 times for 2 min with washing buffer (B1 0.1X+Tween 0.1%) (Eppendorf, Hamburg, Germany).

The micro-arrays were than incubated for 45 min at room temperature with the Cy3-conjugated IgG Anti biotin (Jackson Immuno Research laboratories, Inc #200-162-096) diluted 1/1000× Conjugate-Cy3 in the blocking reagent and protect from light.

The micro-arrays were washed again 5 times for 2 min with washing buffer (B1 0.1X+Tween 0.1%) and 2 times for 2 min with distilled water before being dried under a flux of N₂.

After image acquisition, the scanned 16-bit images are imported to the software, ‘ImaGene4.0’ (BioDiscovery, Los Angeles, Calif., USA), which is used to quantify the signal intensities. The spots intensities are first corrected by a subtraction of the local background intensity from signal intensity.

In order to evaluate the entire experiment, several positive and negative controls (for hybridization and detection) are first analysed. Then the signal obtained on each miRNA spots is analysed in order to correlate the result with the presence or not of miRNA directed against the specific gene in the sample.

TABLE 1

miRNA human sequences

| | | | | | | |
|---|---|---|---|---|---|---|
| ID | Species | Gene | miRNA sequence | Mature | Precursor | SEQ ID NO |

| | | | | | | |
|---|---|---|---|---|---|---|
| hsa-mir-7-1 | Homo sapiens | miR-7-1 | uggaagacuagugauuuuguu | 21 | 110 | 1 |
| hsa-mir-7-2 | Homo sapiens | miR-7-2 | uggaagacuagugauuuuguu | 21 | 110 | 2 |
| hsa-mir-7-3 | Homo sapiens | miR-7-3 | uggaagacuagugauuuuguu | 21 | 110 | 3 |
| hsa-let-7f-2L | Homo sapiens | let-7f-2 | ugagguaguagauuguauaguu | 22 | 89 | 4 |
| hsa-let-7f-1L | Homo sapiens | let-7f-1 | ugagguaguagauuguauaguu | 22 | 87 | 5 |
| hsa-let-7eL | Homo sapiens | let-7e | ugagguaggagguuguauagu | 21 | 79 | 6 |
| hsa-let-7a-1L | Homo sapiens | let-7a-1 | ugagguaguagguuguauaguu | 22 | 80 | 7 |
| hsa-let-7a-2L | Homo sapiens | let-7a-2 | ugagguaguagguuguauaguu | 22 | 72 | 8 |
| hsa-let-7a-3L | Homo sapiens | let-7a-3 | ugagguaguagguuguauaguu | 22 | 74 | 9 |
| hsa-let-7bL | Homo sapiens | let-7b | ugagguaguagguugugugguu | 22 | 83 | 10 |
| hsa-let-7cL | Homo sapiens | let-7c | ugagguaguagguuguaugguu | 22 | 84 | 11 |
| hsa-let-7dL | Homo sapiens | let-7d | agagguaguagguugcauagu | 21 | 87 | 12 |
| hsa-mir-10a | Homo sapiens | mir-10a | uacccuguagauccgaauuugug | 23 | 110 | 13 |
| hsa-mir-10b | Homo sapiens | mir-10b | uacccuguagaaccgaauuugu | 22 | 110 | 14 |
| hsa-mir-15 | Homo sapiens | mir-15 | uagcagcacauaaugguuugug | 22 | 83 | 15 |
| hsa-mir-16 | Homo sapiens | mir-16 | uagcagcacguaaauauuggcg | 22 | 89 | 16 |
| hsa-mir-17 | Homo sapiens | mir-17 | acugcagugaaggcacuugu | 20 | 84 | 17 |
| hsa-mir-18 | Homo sapiens | mir-18 | uaaggugcaucuagugcagaua | 22 | 71 | 18 |
| hsa-mir-19a | Homo sapiens | mir-19a | ugugcaaaucuaugcaaaacuga | 23 | 82 | 19 |
| hsa-mir-19b-1 | Homo sapiens | mir-19b-1 | ugugcaaauccaugcaaaacuga | 23 | 87 | 20 |
| hsa-mir-19b-2 | Homo sapiens | mir-19b-2 | ugugcaaauccaugcaaaacuga | 23 | 96 | 21 |
| hsa-mir-20 | Homo sapiens | mir-20 | uaaagugcuuauagugcaggua | 22 | 71 | 22 |
| hsa-mir-21 | Homo sapiens | mir-21 | uagcuuaucagacugauguuga | 22 | 72 | 23 |
| hsa-mir-22 | Homo sapiens | mir-22 | aagcugccaguugaagaacugu | 22 | 85 | 24 |
| hsa-mir-23 | Homo sapiens | mir-23 | aucacauugccagggauuucc | 21 | 73 | 25 |
| hsa-mir-24-2 | Homo sapiens | mir-24-2 | uggcucaguucagcaggaacag | 22 | 73 | 26 |
| hsa-mir-24-1 | Homo sapiens | mir-24-1 | uggcucaguucagcaggaacag | 22 | 68 | 27 |
| hsa-mir-25 | Homo sapiens | mir-25 | cauugcacuugucucggucuga | 22 | 84 | 28 |
| hsa-mir-26a | Homo sapiens | mir-26a | uucaaguaauccaggauaggcu | 22 | 75 | 29 |
| hsa-mir-26b | Homo sapiens | mir-26b | uucaaguaauucaggauaggu | 21 | 77 | 30 |
| hsa-mir-27 | Homo sapiens | mir-27 | uucacaguggcuaaguuccgcc | 22 | 78 | 31 |
| hsa-mir-28 | Homo sapiens | mir-28 | aaggagcucacagucuauugag | 22 | 86 | 32 |
| hsa-mir-29 | Homo sapiens | mir-29 | cuagcaccaucugaaaucgguu | 22 | 64 | 33 |
| hsa-mir-30c | Homo sapiens | mir-30c | uguaaacauccuacacucucagc | 23 | 72 | 34 |
| hsa-mir-30d | Homo sapiens | mir-30d | uguaaacauccccgacuggaag | 22 | 70 | 35 |
| hsa-mir-30a | Homo sapiens | mir-30a-s | uguaaacauccucgacuggaagc | 23 | 71 | 36 |

The mature sequences miR-30 and miR-97 appear to originate from the same precursor and
the entries have been merged and renamed to match the homologous mouse entry.

| | | | | | | |
|---|---|---|---|---|---|---|
| hsa-mir-30a | Homo sapiens | mir-30a-as | cuuucagucggauguuugcagc | 22 | 71 | 37 |
| hsa-mir-31 | Homo sapiens | mir-31 | ggcaagaugcuggcauagcug | 21 | 71 | 38 |
| hsa-mir-32 | Homo sapiens | mir-32 | uauugcacauuacuaaguugc | 21 | 70 | 39 |
| hsa-mir-33 | Homo sapiens | mir-33 | gugcauuguaguugcauug | 19 | 69 | 40 |
| hsa-mir-34 | Homo sapiens | mir-34 | uggcagugucuuagcugguugu | 22 | 110 | 41 |
| hsa-mir-91 | Homo sapiens | mir-91 | caaagugcuuacagugcagguagu | 24 | 82 | 42 |
| — | Homo sapiens | mir-17 | acugcagugaaggcacuugu | 20 | 82 | 43 |

miR-17 is cleaved from the reverse strand of human precursor mir-91 and from human
precursor mir-17

| | | | | | | |
|---|---|---|---|---|---|---|
| hsa-mir-92-1 | Homo sapiens | mir-92-1 | uauugcacuugucccggccugu | 22 | 78 | 44 |
| hsa-mir-92-2 | Homo sapiens | mir-92-2 | uauugcacuugucccggccugu | 22 | 75 | 45 |
| hsa-mir-93-1 | Homo sapiens | mir-93-1 | aaagugcuguucgugcagguag | 22 | 80 | 46 |
| hsa-mir-93-2 | Homo sapiens | mir-93-2 | aaagugcuguucgugcagguag | 22 | 80 | 47 |
| hsa-mir-95 | Homo sapiens | mir-95 | uucaacggguauuuauugagca | 22 | 81 | 48 |
| hsa-mir-96 | Homo sapiens | mir-96 | uuuggcacuagcacauuuuugc | 22 | 78 | 49 |
| hsa-mir-98 | Homo sapiens | mir-98 | ugagguaguaaguuguauuguu | 22 | 80 | 50 |
| hsa-mir-99 | Homo sapiens | mir-99 | aacccguagauccgaucuugug | 22 | 81 | 51 |
| hsa-mir-100 | Homo sapiens | mir-100 | aacccguagauccgaacuugug | 22 | 80 | 51 |
| hsa-mir-101 | Homo sapiens | mir-101 | uacaguacugugauaacugaag | 22 | 75 | 53 |
| hsa-mir-102-1 | Homo sapiens | mir-102-1 | uagcaccauuugaaaucagu | 20 | 81 | 54 |
| hsa-mir-102-2 | Homo sapiens | mir-102-2 | uagcaccauuugaaaucagu | 20 | 81 | 55 |
| hsa-mir-102-3 | Homo sapiens | mir-102-3 | uagcaccauuugaaaucagu | 20 | 81 | 56 |
| hsa-mir-103-2 | Homo sapiens | mir-103-2 | agcaacauuguacagggcuauga | 23 | 78 | 57 |
| hsa-mir-103-1 | Homo sapiens | mir-103-1 | agcagcauuguacagggcuauga | 23 | 78 | 58 |
| hsa-mir-104 | Homo sapiens | mir-104 | ucaacaucagucugauaagcua | 22 | 78 | 59 |
| hsa-mir-105-1 | Homo sapiens | mir-105-1 | ucaaaugcucagacuccugu | 20 | 81 | 60 |
| hsa-mir-105-2 | Homo sapiens | mir-105-2 | ucaaaugcucagacuccugu | 20 | 81 | 61 |
| hsa-mir-106 | Homo sapiens | mir-106 | aaaagugcuuacagugcagguagc | 24 | 81 | 62 |
| hsa-mir-107 | Homo sapiens | mir-107 | agcagcauuguacagggcuauca | 23 | 81 | 63 |
| hsa-mir-124b | Homo sapiens | mir-124b | uuaaggcacgcggugaaugc | 20 | 67 | 64 |
| hsa-mir-139 | Homo sapiens | mir-139 | ucuacagugcacgugucu | 18 | 68 | 65 |
| hsa-mir-147 | Homo sapiens | mir-147 | guguguggaaaugcuucugc | 20 | 72 | 66 |
| hsa-mir-148 | Homo sapiens | mir-148 | ucagugcacuacagaacuuugu | 22 | 68 | 67 |
| hsa-mir-181c | Homo sapiens | mir-181c | aacauucaaccugucggugagu | 22 | 110 | 68 |
| hsa-mir-181b | Homo sapiens | mir-181b | accaucgaccguugauuguacc | 22 | 110 | 69 |
| hsa-mir-181a | Homo sapiens | mir-181a | aacauucaacgcugucggugagu | 23 | 110 | 70 |
| hsa-mir-182-as | Homo sapiens | mir-182-as | ugguucuagacuugccaacua | 21 | 110 | 71 |
| hsa-mir-183 | Homo sapiens | mir-183 | uauggcacugguagaauucacug | 23 | 110 | 72 |
| hsa-mir-187 | Homo sapiens | mir-187 | ucgugucuuguguugcagccg | 21 | 110 | 73 |
| hsa-mir-192 | Homo sapiens | mir-192 | cugaccuaugaauugacagcc | 21 | 110 | 74 |
| hsa-mir-196-2 | Homo sapiens | mir-196-2 | uagguaguuucauguuguuggg | 22 | 110 | 75 |
| hsa-mir-196-1 | Homo sapiens | mir-196-1 | uagguaguuucauguuguuggg | 22 | 110 | 76 |
| hsa-mir-196 | Homo sapiens | mir-196 | uagguaguuucauguuguugg | 21 | 70 | 77 |
| hsa-mir-197 | Homo sapiens | mir-197 | uucaccaccuucuccacccagc | 22 | 75 | 78 |
| hsa-mir-198 | Homo sapiens | mir-198 | gguccagaggggagauagg | 19 | 62 | 79 |
| hsa-mir-199a-2 | Homo sapiens | mir-199a-2 | cccaguguucagacuaccuguuc | 23 | 110 | 80 |
| hsa-mir-199b | Homo sapiens | mir-199b | cccaguguuuagacuaucuguuc | 23 | 110 | 81 |
| hsa-mir-199a-1 | Homo sapiens | mir-199a-1 | cccaguguucagacuaccuguuc | 23 | 110 | 82 |
| hsa-mir-199-s | Homo sapiens | mir-199-s | cccaguguucagacuaccuguu | 22 | 71 | 83 |
| hsa-mir-200b | Homo sapiens | mir-200b | cucuaauacugccugguaaugaug | 24 | 95 | 84 |
| hsa-mir-203 | Homo sapiens | mir-203 | gugaaauguuuaggaccacuag | 22 | 110 | 85 |
| hsa-mir-204 | Homo sapiens | mir-204 | uucccuuugucauccuaugccu | 22 | 110 | 86 |
| hsa-mir-205 | Homo sapiens | mir-205 | uccuucauuccaccggagucug | 22 | 110 | 87 |
| hsa-mir-208 | Homo sapiens | mir-208 | auaagacgagcaaaaagcuugu | 22 | 71 | 88 |
| hsa-mir-210 | Homo sapiens | mir-210 | cugugcgugugacagcggcug | 21 | 110 | 89 |
| hsa-mir-211 | Homo sapiens | mir-211 | uucccuuugucauccuucgccu | 22 | 110 | 90 |
| hsa-mir-212 | Homo sapiens | mir-212 | uaacagucuccagucacggcc | 21 | 110 | 91 |
| hsa-mir-213 | Homo sapiens | mir-213 | aacauucauugcugucgguggguu | 24 | 110 | 92 |
| hsa-mir-214 | Homo sapiens | mir-214 | acagcaggcacagacaggcag | 21 | 110 | 93 |
| hsa-mir-215 | Homo sapiens | mir-215 | augaccuaugaauugacagac | 21 | 110 | 94 |
| hsa-mir-216 | Homo sapiens | mir-216 | uaaucucagcuggcaacugug | 21 | 110 | 95 |
| hsa-mir-217 | Homo sapiens | mir-217 | uacugcaucaggaacugauuggau | 24 | 110 | 96 |
| hsa-mir-218-1 | Homo sapiens | mir-218-1 | uugugcuugaucuaaccaugu | 21 | 110 | 97 |
| hsa-mir-218-2 | Homo sapiens | mir-218-2 | uugugcuugaucuaaccaugu | 21 | 110 | 98 |
| hsa-mir-219 | Homo sapiens | mir-219 | ugauuguccaaacgcaauucu | 21 | 110 | 99 |
| hsa-mir-220 | Homo sapiens | mir-220 | ccacaccguaucugacacuuu | 21 | 110 | 100 |
| hsa-mir-221 | Homo sapiens | mir-221 | agcuacauugucugcuggguuuc | 23 | 110 | 101 |
| hsa-mir-222 | Homo sapiens | mir-222 | agcuacaucuggcuacugggucuc | 24 | 110 | 102 |
| hsa-mir-223 | Homo sapiens | mir-223 | ugucaguuugucaaauacccc | 21 | 110 | 103 |
| hsa-mir-224 | Homo sapiens | mir-224 | caagucacuagugguuccguuua | 23 | 81 | 104 |

TABLE 2

miRNA mouse sequences

| | | | | | SEQ |
|---|---|---|---|---|---|
| | Spe- | | | Ma- | ID |
| ID | cies | Gene | mirNA sequence | ture | NO |
| mmu- | Mus | mir-1b | UGGAAUGUAAAGAAGUAUGUAA | 22 | 105 |
| mir-1b | mus- | | | | |
| | cu- | | | | |
| | lus | | | | |
| mmu- | Mus | mir-1c | UGGAAUGUAAAGAAGUAUGUAC | 22 | 106 |
| mir-1c | mus- | | | | |
| | cu- | | | | |
| | lus | | | | |
| mmu- | Mus | mir-1d | UGGAAUGUAAAGAAGUAUGUAUU | 23 | 107 |
| mir-1d | mus- | | | | |
| | cu- | | | | |
| | lus | | | | |
| mmu- | Mus | mir-9 | UCUUUGGUUAUCUAGCUGUAUGA | 23 | 108 |
| mir-9 | mus- | | | | |
| | cu- | | | | |
| | lus | | | | |
| mmu- | Mus | mir-9- | UAAAGCUAGAUAACCGAAAGU | 21 | 109 |
| mir-9- | mus- | star | | | |
| star | cu- | | | | |
| | lus | | | | |
| mmu- | Mus | mir- | CCCUGUAGAACCGAAUUUGUGU | 22 | 110 |
| mir- | mus- | 10b | | | |
| 10b | cu- | | | | |
| | lus | | | | |
| mmu- | Mus | mir- | UAGCAGCACAUAAUGGUUUGUG | 22 | 111 |
| mir- | mus- | 15a | | | |
| 15a | cu- | | | | |
| | lus | | | | |
| mmu- | Mus | mir- | UAGCAGCACAUCAUGGUUUACA | 22 | 112 |
| mir- | mus- | 15b | | | |
| 15b | cu- | | | | |
| | lus | | | | |
| mmu- | Mus | mir-16 | UAGCAGCACGUAAAUAUUGGCG | 22 | 113 |
| mir-16 | mus- | | | | |
| | cu- | | | | |
| | lus | | | | |
| mmu- | Mus | mir-18 | UAAGGUGCAUCUAGUGCAGAUA | 22 | 114 |
| mir-18 | mus- | | | | |
| | cu- | | | | |
| | lus | | | | |
| mmu- | Mus | mir- | UGUGCAAAUCCAUGCAAAACUGA | 23 | 115 |
| mir- | mus- | 19b | | | |
| 19b | cu- | | | | |
| | lus | | | | |
| mmu- | Mus | mir-20 | UAAAGUGCUUAUAGUGCAGGUAG | 23 | 116 |
| mir-20 | mus- | | | | |
| | cu- | | | | |
| | lus | | | | |
| mmu- | Mus | mir-21 | UAGCUUAUCAGACUGAUGUUGA | 22 | 117 |
| mir-21 | mus- | | | | |
| | cu- | | | | |
| | lus | | | | |
| mmu- | Mus | mir-22 | AAGCUGCCAGUUGAAGAACUGU | 22 | 118 |
| mir-22 | mus- | | | | |
| | cu- | | | | |
| | lus | | | | |
| mmu- | Mus | mir- | AUCACAUUGCCAGGGAUUUCC | 21 | 119 |
| mir- | mus- | 23a | | | |
| 23a | cu- | | | | |
| | lus | | | | |
| mmu- | Mus | mir- | AUCACAUUGCCAGGGAUUACCAC | 23 | 120 |
| mir- | mus- | 23b | | | |
| 23b | cu- | | | | |
| | lus | | | | |
| mmu- | Mus | mir-24 | UGGCUCAGUUCAGCAGGAACAG | 22 | 121 |
| mir-24 | mus- | | | | |
| | cu- | | | | |
| | lus | | | | |
| mmu- | Mus | mir- | UUCAAGUAAUCCAGGAUAGGCU | 22 | 122 |
| mir- | mus- | 26a | | | |
| 26a | cu- | | | | |
| | lus | | | | |
| mmu- | Mus | mir- | UUCAAGUAAUUCAGGAUAGGUU | 22 | 123 |
| mir- | mus- | 26b | | | |
| 26b | cu- | | | | |
| | lus | | | | |
| mmu- | Mus | mir- | UUCACAGUGGCUAAGUUCCGCU | 22 | 124 |
| mir- | mus- | 27a | | | |
| 27a | cu- | | | | |
| | lus | | | | |
| mmu- | Mus | mir- | UUCACAGUGGCUAAGUUCUG | 20 | 125 |
| mir- | mus- | 27b | | | |
| 27b | cu- | | | | |
| | lus | | | | |
| mmu- | Mus | mir- | CUAGCACCAUCUGAAAUCGGUU | 22 | 126 |
| mir- | mus- | 29a | | | |
| 29a | cu- | | | | |
| | lus | | | | |
| mmu- | Mus | mir- | UAGCACCAUUUGAAAUCAGUGUU | 23 | 127 |
| mir- | mus- | 29b | | | |
| 29b | cu- | | | | |
| | lus | | | | |
| mmu- | Mus | mir- | UAGCACCAUUUGAAAUCGGUUA | 22 | 128 |
| mir- | mus- | 29c | | | |
| 29c | cu- | | | | |
| | lus | | | | |
| mmu- | Mus | mir- | UGUAAACAUCCUCGACUGGAAGC | 23 | 129 |
| mir- | mus- | 30a | | | |
| 30a | cu- | | | | |
| | lus | | | | |
| mmu- | Mus | mir- | CUUUCAGUCGGAUGUUUGCAGC | 22 | 130 |
| mir- | mus- | 30a-as | | | |
| 30a-as | cu- | | | | |
| | lus | | | | |
| mmu- | Mus | mir- | UGUAAACAUCCUACACUCAGC | 21 | 131 |
| mir- | mus- | 30b | | | |
| 30bb | cu- | | | | |
| | lus | | | | |
| mmu- | Mus | mir- | UGUAAACAUCCUACACUCUCAGC | 23 | 132 |
| mir- | mus- | 30c | | | |
| 30c | cu- | | | | |
| | lus | | | | |
| mmu- | Mus | mir- | UGUAAACAUCCCCGACUGGAAG | 22 | 133 |
| mir- | mus- | 30d | | | |
| 30d | cu- | | | | |
| | lus | | | | |
| mmu- | Mus | mir- | ACCCGUAGAUCCGAUCUUGU | 20 | 134 |
| mir- | mus- | 99a | | | |
| 99a | cu- | | | | |
| | lus | | | | |
| mmu- | Mus | mir- | CACCCGUAGAACCGACCUUGCG | 22 | 135 |
| mir- | mus- | 99b | | | |
| 99b | cu- | | | | |
| | lus | | | | |
| mmu- | Mus | mir- | UACAGUACUGUGAUAACUGA | 20 | 136 |
| mir- | mus- | 101 | | | |
| 101 | cu- | | | | |
| | lus | | | | |
| mmu- | Mus | mir- | UGGAGUGUGACAAUGGUGUUUGU | 23 | 137 |
| mir- | mus- | 122a | | | |
| 122a | cu- | | | | |
| | lus | | | | |
| mmu- | Mus | mir- | UGGAGUGUGACAAUGGUGUUUGA | 23 | 138 |
| mir- | mus- | 122b | | | |
| 122b | cu- | | | | |
| | lus | | | | |
| mmu- | Mus | mir- | UUAAGGCACGCGGUGAAUGCCA | 22 | 139 |
| mir- | mus- | 124a | | | |
| 124a | cu- | | | | |
| | lus | | | | |
| mmu- | Mus | mir- | UUAAGGCACGCGGGUGAAUGC | 21 | 140 |
| mir- | mus- | 124b | | | |
| 124b | cu- | | | | |
| | lus | | | | |
| mmu- | Mus | mir- | UCCCUGAGACCCUUUAACCUGUG | 23 | 141 |
| mir- | mus- | 125a | | | |
| 125a | cu- | | | | |
| | lus | | | | |
| mmu- | Mus | mir- | UCCCUGAGACCCUAACUUGUGA | 22 | 142 |
| mir- | mus- | 125b | | | |
| 125b | cu- | | | | |
| | lus | | | | |
| mmu- | Mus | mir- | UCGUACCGUGAGUAAUAAUGC | 21 | 143 |
| mir- | mus- | 126 | | | |
| 126 | cu- | | | | |
| | lus | | | | |
| mmu- | Mus | mir- | CAUUAUUACUUUUGGUACGCG | 21 | 144 |
| mir- | mus- | 126- | | | |
| 126- | cu- | star | | | |
| star | lus | | | | |
| mmu- | Mus | mir- | UCGGAUCCGUCUGAGCUUGGCU | 22 | 145 |
| mir- | mus- | 127 | | | |
| 127 | cu- | | | | |
| | lus | | | | |
| mmu- | Mus | mir- | UCACAGUGAACCGGUCUCUUUU | 22 | 146 |
| mir- | mus- | 128 | | | |
| 128 | cu- | | | | |
| | lus | | | | |
| mmu- | Mus | mir- | CUUUUUUCGGUCUGGGCUUGC | 21 | 147 |
| mir- | mus- | 129 | | | |
| 129 | cu- | | | | |
| | lus | | | | |
| mmu- | Mus | mir- | CUUUUUGCGGUCUGGGCUUGCU | 22 | 148 |
| mir- | mus- | 129b | | | |
| 129b | cu- | | | | |
| | lus | | | | |
| mmu- | Mus | mir- | CAGUGCAAUGUUAAAAGGGC | 20 | 149 |
| mir- | mus- | 130 | | | |
| 130 | cu- | | | | |
| | lus | | | | |
| mmu- | Mus | mir- | UAACAGUCUACAGCCAUGGUCGU | 23 | 150 |
| mir- | mus- | 132 | | | |
| 132 | cu- | | | | |
| | lus | | | | |
| mmu- | Mus | mir- | UUGGUCCCCUUCAACCAGCUGU | 22 | 151 |
| mir- | mus- | 133 | | | |
| 133 | cu- | | | | |
| | lus | | | | |
| mmu- | Mus | mir- | UGUGACUGGUUGACCAGAGGGA | 22 | 152 |
| mir- | mus- | 134 | | | |
| 134 | cu- | | | | |
| | lus | | | | |
| mmu- | Mus | mir- | UAUGGCUUUUUAUUCCUAUGUGAA | 24 | 153 |
| mir- | mus- | 135 | | | |
| 135 | cu- | | | | |
| | lus | | | | |
| mmu- | Mus | mir- | ACUCCAUUUGUUUUGAUGAUGGA | 23 | 154 |
| mir- | mus- | 136 | | | |
| 136 | cu- | | | | |
| | lus | | | | |
| mmu- | Mus | mir- | UAUUGCUUAAGAAUACGCGUAG | 22 | 155 |
| mir- | mus- | 137 | | | |
| 137 | cu- | | | | |
| | lus | | | | |
| mmu- | Mus | mir- | AGCUGGUGUUGUGAAUC | 17 | 156 |
| mir- | mus- | 138 | | | |
| 138 | cu- | | | | |
| | lus | | | | |
| mmu- | Mus | mir- | UCUACAGUGCACGUGUCU | 18 | 157 |
| mir- | mus- | 139 | | | |
| 139 | cu- | | | | |
| | lus | | | | |
| mmu- | Mus | mir- | AGUGGUUUUACCCUAUGGUAG | 21 | 158 |
| mir- | mus- | 140 | | | |
| 140 | cu- | | | | |
| | lus | | | | |
| mmu- | Mus | mir- | AACACUGUCUGGUAAAGAUGG | 21 | 159 |
| mir- | mus- | 141 | | | |
| 141 | cu- | | | | |
| | lus | | | | |
| mmu- | Mus | mir- | CAUAAAGUAGAAAGCACUAC | 20 | 160 |
| mir- | mus- | 142s | | | |
| 142s | cu- | | | | |
| | lus | | | | |
| mmu- | Mus | mir- | UGUAGUGUUUCCUACUUUAUGG | 22 | 161 |
| mir- | mus- | 142as | | | |
| 142as | cu- | | | | |
| | lus | | | | |
| mmu- | Mus | mir- | UGAGAUGAAGCACUGUAGCUCA | 22 | 162 |
| mir- | mus- | 143 | | | |
| 143 | cu- | | | | |
| | lus | | | | |
| mmu- | Mus | mir- | UACAGUAUAGAUGAUGUACUAG | 22 | 163 |
| mir- | mus- | 144 | | | |
| 144 | cu- | | | | |
| | lus | | | | |
| mmu- | Mus | mir- | GUCCAGUUUUCCCAGGAAUCCCUU | 24 | 164 |
| mir- | mus- | 145 | | | |
| 145 | cu- | | | | |
| | lus | | | | |
| mmu- | Mus | mir- | UGAGAACUGAAUUCCAUGGGUUU | 23 | 165 |
| mir- | mus- | 146 | | | |
| 146 | cu- | | | | |
| | lus | | | | |
| mmu- | Mus | mir- | GUGUGUGGAAAUGCUUCUGCC | 21 | 166 |
| mir- | mus- | 147 | | | |
| 147 | cu- | | | | |
| | lus | | | | |
| mmu- | Mus | mir- | UCAGUGCACUACAGAACUUUGU | 22 | 167 |
| mir- | mus- | 148 | | | |
| 148 | cu- | | | | |
| | lus | | | | |
| mmu- | Mus | mir- | UCUGGCUCCGUGUCUUCACUCC | 22 | 168 |
| mir- | mus- | 149 | | | |
| 149 | cu- | | | | |
| | lus | | | | |
| mmu- | Mus | mir- | UCUCCCAACCCUUGUACCAGUGU | 23 | 169 |
| mir- | mus- | 150 | | | |
| 150 | cu- | | | | |
| | lus | | | | |
| mmu- | Mus | mir- | CUAGACUGAGGCUCCUUGAGGU | 22 | 170 |
| mir- | mus- | 151 | | | |
| 151 | cu- | | | | |
| | lus | | | | |
| mmu- | Mus | mir- | UCAGUGCAUGACAGAACUUGG | 21 | 171 |
| mir- | mus- | 152 | | | |
| 152 | cu- | | | | |
| | lus | | | | |
| mmu- | Mus | mir- | UUGCAUAGUCACAAAAGUGA | 20 | 172 |
| mir- | mus- | 153 | | | |
| 153 | cu- | | | | |
| | lus | | | | |
| mmu- | Mus | mir- | UAGGUUAUCCGUGUUGCCUUCG | 22 | 173 |
| mir- | mus- | 154 | | | |
| 154 | cu- | | | | |
| | lus | | | | |
| mmu- | Mus | mir- | UUAAUGCUAAUUGUGAUAGGGG | 22 | 174 |
| mir- | mus- | 155 | | | |
| 155 | cu- | | | | |
| | lus | | | | |
| mmu- | Mus | mir- | AACAUUCAACGCUGUCGGUGAGU | 23 | 175 |
| mir- | mus- | 181 | | | |
| 181 | cu- | | | | |
| | lus | | | | |
| mmu- | Mus | mir- | UUUGGCAAUGGUAGAACUCACA | 22 | 176 |
| mir- | mus- | 182 | | | |
| 182 | cu- | | | | |
| | lus | | | | |
| mmu- | Mus | mir- | UAUGGCACUGGUAGAAUUCACUG | 23 | 177 |
| mir- | mus- | 183 | | | |
| 183 | cu- | | | | |
| | lus | | | | |
| mmu- | Mus | mir- | UGGACGGAGAACUGAUAAGGGU | 22 | 178 |
| mir- | mus- | 184 | | | |
| 184 | cu- | | | | |
| | lus | | | | |
| mmu- | Mus | mir- | UGGAGAGAAAGGCAGUUC | 18 | 179 |
| mir- | mus- | 185 | | | |
| 185 | cu- | | | | |
| | lus | | | | |
| mmu- | Mus | mir- | CAAAGAAUUCUCCUUUUGGGCUU | 23 | 180 |
| mir- | mus- | 186 | | | |
| 186 | cu- | | | | |
| | lus | | | | |
| mmu- | Mus | mir- | UCGUGUCUUGUGUUGCAGCCGG | 22 | 181 |
| mir- | mus- | 187 | | | |
| 187 | cu- | | | | |
| | lus | | | | |
| mmu- | Mus | mir- | CAUCCCUUGCAUGGUGGAGGGU | 22 | 182 |
| mir- | mus- | 188 | | | |
| 188 | cu- | | | | |
| | lus | | | | |
| mmu- | Mus | mir- | GUGCCUACUGAGCUGACAUCAGU | 23 | 183 |
| mir- | mus- | 189 | | | |
| 189 | cu- | | | | |
| | lus | | | | |
| mmu- | Mus | mir- | UGAUAUGUUUGAUAUAUUAGGU | 22 | 184 |
| mir- | mus- | 190 | | | |
| 190 | cu- | | | | |
| | lus | | | | |
| mmu- | Mus | mir- | CAACGGAAUCCCAAAAGCAGCU | 22 | 185 |
| mir- | mus- | 191 | | | |
| 191 | cu- | | | | |
| | lus | | | | |
| mmu- | Mus | mir- | CUGACCUAUGAAUUGACA | 18 | 186 |
| mir- | mus- | 192 | | | |
| 192 | cu- | | | | |
| | lus | | | | |
| mmu- | Mus | mir- | AACUGGCCUACAAAGUCCCAG | 21 | 187 |
| mir- | mus- | 193 | | | |
| 193 | cu- | | | | |
| | lus | | | | |
| mmu- | Mus | mir- | UGUAACAGCAACUCCAUGUGGA | 22 | 188 |
| mir- | mus- | 194 | | | |
| 194 | cu- | | | | |
| | lus | | | | |
| mmu- | Mus | mir- | UAGCAGCACAGAAAUAUUGGC | 21 | 189 |
| mir- | mus- | 195 | | | |
| 195 | cu- | | | | |
| | lus | | | | |
| mmu- | Mus | mir- | UAGGUAGUUUCAUGUUGUUGG | 21 | 190 |
| mir- | mus- | 196 | | | |
| 196 | cu- | | | | |
| | lus | | | | |
| mmu- | Mus | mir- | CCCAGUGUUCAGACUACCUGUU | 22 | 191 |
| mir- | mus- | 199s | | | |
| 199 | cu- | | | | |
| | lus | | | | |
| mmu- | Mus | mir- | UACAGUAGUCUGCACAUUGGUU | 22 | 192 |
| mir- | mus- | 199as | | | |
| 199as | cu- | | | | |
| | lus | | | | |
| mmu- | Mus | mir- | UAACACUGUCUGGUAACGAUGU | 22 | 193 |
| mir- | mus- | 200a | | | |
| 200a | cu- | | | | |
| | lus | | | | |
| mmu- | Mus | mir- | UAAUACUGCCUGGUAAUGAUGAC | 23 | 194 |
| mir- | mus- | 200b | | | |
| 200b | cu- | | | | |
| | lus | | | | |
| mmu- | Mus | mir- | UACUCAGUAAGGCAUUGUUCU | 21 | 195 |
| mir- | mus- | 201 | | | |
| 201 | cu- | | | | |
| | lus | | | | |
| mmu- | Mus | mir- | AGAGGUAUAGCGCAUGGGAAGA | 22 | 196 |
| mir- | mus- | 202 | | | |
| 202 | cu- | | | | |
| | lus | | | | |
| mmu- | Mus | mir- | GUGAAAUGUUUAGGACCACUAGA | 23 | 197 |
| mir- | mus- | 203 | | | |
| 203 | cu- | | | | |
| | lus | | | | |
| mmu- | Mus | mir- | UUCCCUUUGUCAUCCUAUGCCUG | 23 | 198 |
| mir- | mus- | 204 | | | |
| 204 | cu- | | | | |
| | lus | | | | |
| mmu- | Mus | mir- | UCCUUCAUUCCACCGGAGUCUG | 22 | 199 |
| mir- | mus- | 205 | | | |
| 205 | cu- | | | | |
| | lus | | | | |
| mmu- | Mus | mir- | UGGAAUGUAAGGAAGUGUGUGG | 22 | 200 |
| mir- | mus- | 206 | | | |
| 206 | cu- | | | | |
| | lus | | | | |
| mmu- | Mus | mir- | GCUUCUCCUGGCUCUCCUCCCUC | 23 | 201 |
| mir- | mus- | 207 | | | |
| 207 | cu- | | | | |
| | lus | | | | |
| mmu- | Mus | mir- | AUAAGACGAGCAAAAAGCUUGU | 22 | 202 |
| mir- | mus- | 208 | | | |
| 208 | cu- | | | | |
| | lus | | | | |
| mmu- | Mus | let-7a | UGAGGUAGUAGGUUGUGUGGUU | 22 | 203 |
| let-7a | mus- | | | | |
| | cu- | | | | |
| | lus | | | | |
| mmu- | Mus | let-7b | UGAGGUAGUAGGUUGUAUAGUU | 22 | 204 |
| let-7b | mus- | | | | |
| | cu- | | | | |
| | lus | | | | |
| mmu- | Mus | let-7c | UGAGGUAGUAGGUUGUAUGGUU | 22 | 205 |
| let-7c | mus- | | | | |
| | cu- | | | | |
| | lus | | | | |
| mmu- | Mus | let-7d | AGAGGUAGUAGGUUGCAUAGU | 21 | 206 |
| let-7d | mus- | | | | |
| | cu- | | | | |
| | lus | | | | |
| mmu- | Mus | let-7e | UGAGGUAGGAGGUUGUAUAGU | 21 | 207 |
| let-7e | mus- | | | | |
| | cu- | | | | |
| | lus | | | | |
| mmu- | Mus | let- | UGAGGUAGUAGAUUGUAUAGUU | 22 | 208 |
| let- | mus- | 7f-1 | | | |
| 7f-1 | cu- | | | | |
| | lus | | | | |
| mmu- | Mus | let- | UGAGGUAGUAGAUUGUAUAGUU | 22 | 209 |
| let- | mus- | 7f-2 | | | |
| 7f-2 | cu- | | | | |
| | lus | | | | |
| mmu- | Mus | let-7g | UGAGGUAGUAGUUUGUACAGUA | 22 | 210 |
| let-7g | mus- | | | | |
| | cu- | | | | |
| | lus | | | | |
| mmu- | Mus | let-7h | UGAGGUAGUAGUGUGUACAGUU | 22 | 211 |
| let-7h | mus- | | | | |
| | cu- | | | | |
| | lus | | | | |
| mmu- | Mus | let-7i | UGAGGUAGUAGUUUGUGCU | 19 | 212 |
| let-7i | mus- | | | | |
| | cu- | | | | |
| | lus | | | | |

What is claimed is:

1. A method for determining the RNAi mediated transcriptional regulation in a cell by the determination of a pattern of at least 3 miRNA detected simultaneously and quantified in the same cell extract, the method comprising the steps of:
(i) providing an array onto which capture probes, reflecting the genomic or transcriptional matter of a cell, are arranged on pre-determined locations thereof;
(ii) isolating a miRNA pool potentially present from a cell;
(iii) elongating or ligating said miRNAs into target labeled polynucleotides;
(iv) contacting said target labeled polynucleotides with the array under conditions allowing hybridization of the target labeled polynucleotides to complementary capture probes present on the array;
detecting and quantifying a signal present on a specific location on the array; wherein the detection of a pattern of at least 3 signals on the array reflects the pattern of miRNAs being involved in the RNAi mediated cellular transcriptional regulation.

2. The method of claim 1, wherein the RNAi mediated cellular transcriptional regulation provided by the detection and quantification of a pattern of miRNAs is correlated with the pattern of expression of the regulated genes in the same sample.

3. The method of claim 1, wherein the RNAi mediated cellular transcriptional regulation provided by the detection and quantification of a pattern of miRNAs is correlated with the pattern of expression of the miRNA targeted genes in the same sample.

4. The method of claim 1, wherein the RNAi mediated cellular transcriptional regulation provided by the detection and quantification of a pattern of miRNAs is correlated with the pattern of expression of the genes having mRNA sequences complementary to the corresponding miRNA sequences in the same sample.

5. The method of claim 1, wherein the RNAi mediated cellular transcriptional regulation is related to the development of an organism.

6. The method of claim 1, wherein the RNAi mediated cellular transcriptional regulation is related to cell differentiation or stem cell maintenance.

7. The method of claim 1 wherein the RNAi mediated cellular transcriptional regulation is related to cell proliferation.

8. The method of claim 1, wherein the RNAi mediated cellular transcriptional regulation is related to cell death.

9. The method of claim 1, wherein the RNAi mediated cellular transcriptional regulation is related to chromatin condensation.

10. The method of claim 1, wherein the RNAi mediated cellular transcriptional regulation is related to cell transformation.

11. The method of claim 1, wherein the miRNA is incorporated into a labeled DNA-RNA sequence which is then detected on the array.

12. The method of claim 1, wherein elongation of the miRNA hybridized on its complementary bait sequence is effected with the Tth DNA polymerase 3.

13. The method of claim 1, wherein elongation of the miRNA hybridized on its complementary bait sequence is effected with the AMV reverse transcriptase.

14. The method of claim 1, wherein elongation of the miRNA hybridized on its complementary bait sequence is effected with the M-MLV reverse transcriptase.

15. The method of claim 1, wherein ligation of the miRNA hybridized on its complementary bait sequence is affected by ligation with an adjacent probe.

16. The method of claim 15, wherein the adjacent probe is pre-hybridized with its complementary sequence before ligation with the miRNA.

17. The method of claim 15, wherein ligation of the miRNA with the adjacent probe is effected with the T4 RNA ligase.

18. The method of claim 15, wherein the adjacent probe is labeled.

19. The method of any one of claims 11 through 14, wherein the elongation of the miRNA is effected on a sequence comprising three parts, the 3′ end is complementary of the miRNA, the middle part is specific of each bait and the 5′ end sequence is common to all baits.

20. The method of claim 19, wherein the elongated miRNAs are amplified.

21. The method of claim 20, wherein the amplification is performed after miRNA degradation using as matrix for the amplification a DNA/DNA hybrid complex.

22. The method of claim 19, wherein a primer complementary of the common sequence of the elongated DNA is provided for amplification.

23. The method of claim 22, wherein the amplification is performed with a DNA polymerase.

24. The method of claim 22, wherein the primer comprises a T7 promoter sequence for an RNA polymerase.

25. The method of claim 22, wherein the primer comprises a Tag sequence.

26. The method of claims 24 or 25, wherein the primer is used for in vitro transcription with a RNA polymerase.

27. The method of claim 1, wherein the array comprises capture probes ranging from about 15 to about 1000 nucleotides, preferably from about 15 to about 200, or 15 to 100 nucleotides.

28. The method of claim 1, wherein the array comprises 5-500 and preferably 5-5000 capture probes.

29. The method of claim 1, wherein the signals present on the array correspond to a pattern of at least 10 miRNAs, preferably at least 20 miRNAs.

30. The method of claims 27 or 28, wherein the capture probes have sequences which are identical for at least 10 to 1000 nucleotides to same part of the mRNA corresponding to the miRNA to be detected.

31. A kit for the determination of RNAi mediated cellular transcriptional regulation in a sample comprising an array comprising at least 3 capture probes being arranged on pre-determined locations and reflecting the genomic or transcriptional matter of a cell and optionally, buffers and labels.

32. A kit for the determination of RNAi mediated cellular transcriptional regulation in a sample comprising two arrays comprising at least 3 capture probes being arranged on pre-determined locations and reflecting the genomic or transcriptional matter of a cell, wherein the first array is dedicated to the detection and of multiple miRNAs present and the second array is dedicated to the detection and quantification of the expression of the regulated genes in the same sample and optionally, buffers and labels.

33. A kit of claim 32, wherein the two arrays are present on the same support.

34. A kit of claim 32, wherein the two arrays are present on the different supports.

35. A kit of claim 32, wherein the capture probes of the array for the detection of the miRNAs and of the mRNA are nucleotide sequences having part of their sequence identical to the mRNA.


SEQUENCE LISTING (212 sequences)

SEQ ID NO: 1 - RNA, 21 nt, Homo Sapiens
uggaagacuagugauuuuguu

SEQ ID NO: 2 - RNA, 21 nt, Homo Sapiens
uggaagacuagugauuuuguu

SEQ ID NO: 3 - RNA, 21 nt, Homo Sapiens
uggaagacuagugauuuuguu

SEQ ID NO: 4 - RNA, 22 nt, Homo Sapiens
ugagguaguagauuguauaguu

SEQ ID NO: 5 - RNA, 22 nt, Homo Sapiens
ugagguaguagauuguauaguu

SEQ ID NO: 6 - RNA, 21 nt, Homo Sapiens
ugagguaggagguuguauagu

SEQ ID NO: 7 - RNA, 22 nt, Homo Sapiens
ugagguaguagguuguauaguu

SEQ ID NO: 8 - RNA, 22 nt, Homo Sapiens
ugagguaguagguuguauaguu

SEQ ID NO: 9 - RNA, 22 nt, Homo Sapiens
ugagguaguagguuguauaguu

SEQ ID NO: 10 - RNA, 22 nt, Homo Sapiens
ugagguaguagguugugugguu

SEQ ID NO: 11 - RNA, 22 nt, Homo Sapiens
ugagguaguagguuguaugguu

SEQ ID NO: 12 - RNA, 21 nt, Homo Sapiens
agagguaguagguugcauagu

SEQ ID NO: 13 - RNA, 23 nt, Homo Sapiens
uacccuguagauccgaauuugug

SEQ ID NO: 14 - RNA, 22 nt, Homo Sapiens
uacccuguagaaccgaauuugu

SEQ ID NO: 15 - RNA, 22 nt, Homo Sapiens
uagcagcacauaaugguuugug

SEQ ID NO: 16 - RNA, 22 nt, Homo Sapiens
uagcagcacguaaauauuggcg

SEQ ID NO: 17 - RNA, 20 nt, Homo Sapiens
acugcagugaaggcacuugu

SEQ ID NO: 18 - RNA, 22 nt, Homo Sapiens
uaaggugcaucuagugcagaua

SEQ ID NO: 19 - RNA, 23 nt, Homo Sapiens
ugugcaaaucuaugcaaaacuga

SEQ ID NO: 20 - RNA, 23 nt, Homo Sapiens
ugugcaaauccaugcaaaacuga

SEQ ID NO: 21 - RNA, 23 nt, Homo Sapiens
ugugcaaauccaugcaaaacuga

SEQ ID NO: 22 - RNA, 22 nt, Homo Sapiens
uaaagugcuuauagugcaggua

SEQ ID NO: 23 - RNA, 22 nt, Homo Sapiens
uagcuuaucagacugauguuga

SEQ ID NO: 24 - RNA, 22 nt, Homo Sapiens
aagcugccaguugaagaacugu

SEQ ID NO: 25 - RNA, 21 nt, Homo Sapiens
aucacauugccagggauuucc

SEQ ID NO: 26 - RNA, 22 nt, Homo Sapiens
uggcucaguucagcaggaacag

SEQ ID NO: 27 - RNA, 22 nt, Homo Sapiens
uggcucaguucagcaggaacag

SEQ ID NO: 28 - RNA, 22 nt, Homo Sapiens
cauugcacuugucucggucuga

SEQ ID NO: 29 - RNA, 22 nt, Homo Sapiens
uucaaguaauccaggauaggcu

SEQ ID NO: 30 - RNA, 21 nt, Homo Sapiens
uucaaguaauucaggauaggu

SEQ ID NO: 31 - RNA, 22 nt, Homo Sapiens
uucacaguggcuaaguuccgcc

SEQ ID NO: 32 - RNA, 22 nt, Homo Sapiens
aaggagcucacagucuauugag

SEQ ID NO: 33 - RNA, 22 nt, Homo Sapiens
cuagcaccaucugaaaucgguu

SEQ ID NO: 34 - RNA, 23 nt, Homo Sapiens
uguaaacauccuacacucucagc

SEQ ID NO: 35 - RNA, 22 nt, Homo Sapiens
uguaaacauccccgacuggaag

SEQ ID NO: 36 - RNA, 23 nt, Homo Sapiens
uguaaacauccucgacuggaagc

SEQ ID NO: 37 - RNA, 22 nt, Homo Sapiens
cuuucagucggauguuugcagc

SEQ ID NO: 38 - RNA, 21 nt, Homo Sapiens
ggcaagaugcuggcauagcug

SEQ ID NO: 39 - RNA, 21 nt, Homo Sapiens
uauugcacauuacuaaguugc

SEQ ID NO: 40 - RNA, 19 nt, Homo Sapiens
gugcauuguaguugcauug

SEQ ID NO: 41 - RNA, 22 nt, Homo Sapiens
uggcagugucuuagcugguugu

SEQ ID NO: 42 - RNA, 24 nt, Homo Sapiens
caaagugcuuacagugcagguagu

SEQ ID NO: 43 - RNA, 20 nt, Homo Sapiens
acugcagugaaggcacuugu

SEQ ID NO: 44 - RNA, 22 nt, Homo Sapiens
uauugcacuugucccggccugu

SEQ ID NO: 45 - RNA, 22 nt, Homo Sapiens
uauugcacuugucccggccugu

SEQ ID NO: 46 - RNA, 22 nt, Homo Sapiens
aaagugcuguucgugcagguag

SEQ ID NO: 47 - RNA, 22 nt, Homo Sapiens
aaagugcuguucgugcagguag

SEQ ID NO: 48 - RNA, 22 nt, Homo Sapiens
uucaacggguauuuauugagca

SEQ ID NO: 49 - RNA, 22 nt, Homo Sapiens
uuuggcacuagcacauuuuugc

SEQ ID NO: 50 - RNA, 22 nt, Homo Sapiens
ugagguaguaaguuguauuguu

SEQ ID NO: 51 - RNA, 22 nt, Homo Sapiens
aacccguagauccgaucuugug

SEQ ID NO: 52 - RNA, 22 nt, Homo Sapiens
aacccguagauccgaacuugug

SEQ ID NO: 53 - RNA, 22 nt, Homo Sapiens
uacaguacugugauaacugaag

SEQ ID NO: 54 - RNA, 20 nt, Homo Sapiens
uagcaccauuugaaaucagu

SEQ ID NO: 55 - RNA, 20 nt, Homo Sapiens
uagcaccauuugaaaucagu

SEQ ID NO: 56 - RNA, 20 nt, Homo Sapiens
uagcaccauuugaaaucagu

SEQ ID NO: 57 - RNA, 23 nt, Homo Sapiens
agcaacauuguacagggcuauga

SEQ ID NO: 58 - RNA, 23 nt, Homo Sapiens
agcagcauuguacagggcuauga

SEQ ID NO: 59 - RNA, 22 nt, Homo Sapiens
ucaacaucagucugauaagcua

SEQ ID NO: 60 - RNA, 20 nt, Homo Sapiens
ucaaaugcucagacuccugu

SEQ ID NO: 61 - RNA, 20 nt, Homo Sapiens
ucaaaugcucagacuccugu

SEQ ID NO: 62 - RNA, 24 nt, Homo Sapiens
aaaagugcuuacagugcagguagc

SEQ ID NO: 63 - RNA, 23 nt, Homo Sapiens
agcagcauuguacagggcuauca

SEQ ID NO: 64 - RNA, 20 nt, Homo Sapiens
uuaaggcacgcggugaaugc

SEQ ID NO: 65 - RNA, 18 nt, Homo Sapiens
ucuacagugcacgugucu

SEQ ID NO: 66 - RNA, 20 nt, Homo Sapiens
guguguggaaaugcuucugc

SEQ ID NO: 67 - RNA, 22 nt, Homo Sapiens
ucagugcacuacagaacuuugu

SEQ ID NO: 68 - RNA, 22 nt, Homo Sapiens
aacauucaaccugucggugagu

SEQ ID NO: 69 - RNA, 22 nt, Homo Sapiens
accaucgaccguugauuguacc

SEQ ID NO: 70 - RNA, 23 nt, Homo Sapiens
aacauucaacgcugucggugagu

SEQ ID NO: 71 - RNA, 21 nt, Homo Sapiens
ugguucuagacuugccaacua

SEQ ID NO: 72 - RNA, 23 nt, Homo Sapiens
uauggcacugguagaauucacug

SEQ ID NO: 73 - RNA, 21 nt, Homo Sapiens
ucgugucuuguguugcagccg

SEQ ID NO: 74 - RNA, 21 nt, Homo Sapiens
cugaccuaugaauugacagcc

SEQ ID NO: 75 - RNA, 22 nt, Homo Sapiens
uagguaguuucauguuguuggg

SEQ ID NO: 76 - RNA, 22 nt, Homo Sapiens
uagguaguuucauguuguuggg

SEQ ID NO: 77 - RNA, 21 nt, Homo Sapiens
uagguaguuucauguuguugg

SEQ ID NO: 78 - RNA, 22 nt, Homo Sapiens
uucaccaccuucuccacccagc

SEQ ID NO: 79 - RNA, 19 nt, Homo Sapiens
gguccagaggggagauagg

SEQ ID NO: 80 - RNA, 23 nt, Homo Sapiens
cccaguguucagacuaccuguuc

SEQ ID NO: 81 - RNA, 23 nt, Homo Sapiens
cccaguguuuagacuaucuguuc

SEQ ID NO: 82 - RNA, 23 nt, Homo Sapiens
cccaguguucagacuaccuguuc

SEQ ID NO: 83 - RNA, 22 nt, Homo Sapiens
cccaguguucagacuaccuguu

SEQ ID NO: 84 - RNA, 24 nt, Homo Sapiens
cucuaauacugccugguaaugaug

SEQ ID NO: 85 - RNA, 22 nt, Homo Sapiens
gugaaauguuuaggaccacuag

SEQ ID NO: 86 - RNA, 22 nt, Homo Sapiens
uucccuuugucauccuaugccu

SEQ ID NO: 87 - RNA, 22 nt, Homo Sapiens
uccuucauuccaccggagucug

SEQ ID NO: 88 - RNA, 22 nt, Homo Sapiens
auaagacgagcaaaaagcuugu

SEQ ID NO: 89 - RNA, 21 nt, Homo Sapiens
cugugcgugugacagcggcug

SEQ ID NO: 90 - RNA, 22 nt, Homo Sapiens
uucccuuugucauccuucgccu

SEQ ID NO: 91 - RNA, 21 nt, Homo Sapiens
uaacagucuccagucacggcc

SEQ ID NO: 92 - RNA, 24 nt, Homo Sapiens
aacauucauugcugucgguggguu

SEQ ID NO: 93 - RNA, 21 nt, Homo Sapiens
acagcaggcacagacaggcag

SEQ ID NO: 94 - RNA, 21 nt, Homo Sapiens
augaccuaugaauugacagac

SEQ ID NO: 95 - RNA, 21 nt, Homo Sapiens
uaaucucagcuggcaacugug

SEQ ID NO: 96 - RNA, 24 nt, Homo Sapiens
uacugcaucaggaacugauuggau

SEQ ID NO: 97 - RNA, 21 nt, Homo Sapiens
uugugcuugaucuaaccaugu

SEQ ID NO: 98 - RNA, 21 nt, Homo Sapiens
uugugcuugaucuaaccaugu

SEQ ID NO: 99 - RNA, 21 nt, Homo Sapiens
ugauuguccaaacgcaauucu

SEQ ID NO: 100 - RNA, 21 nt, Homo Sapiens
ccacaccguaucugacacuuu

SEQ ID NO: 101 - RNA, 23 nt, Homo Sapiens
agcuacauugucugcuggguuuc

SEQ ID NO: 102 - RNA, 24 nt, Homo Sapiens
agcuacaucuggcuacugggucuc

SEQ ID NO: 103 - RNA, 21 nt, Homo Sapiens
ugucaguuugucaaauacccc

SEQ ID NO: 104 - RNA, 23 nt, Homo Sapiens
caagucacuagugguuccguuua

SEQ ID NO: 105 - RNA, 22 nt, Mus musculus
uggaauguaaagaaguauguaa

SEQ ID NO: 106 - RNA, 22 nt, Mus musculus
uggaauguaaagaaguauguac

SEQ ID NO: 107 - RNA, 23 nt, Mus musculus
uggaauguaaagaaguauguauu

SEQ ID NO: 108 - RNA, 23 nt, Mus musculus
ucuuugguuaucuagcuguauga

SEQ ID NO: 109 - RNA, 21 nt, Mus musculus
uaaagcuagauaaccgaaagu

SEQ ID NO: 110 - RNA, 22 nt, Mus musculus
cccuguagaaccgaauuugugu

SEQ ID NO: 111 - RNA, 22 nt, Mus musculus
uagcagcacauaaugguuugug

SEQ ID NO: 112 - RNA, 22 nt, Mus musculus
uagcagcacaucaugguuuaca

SEQ ID NO: 113 - RNA, 22 nt, Mus musculus
uagcagcacguaaauauuggcg

SEQ ID NO: 114 - RNA, 22 nt, Mus musculus
uaaggugcaucuagugcagaua

SEQ ID NO: 115 - RNA, 23 nt, Mus musculus
ugugcaaauccaugcaaaacuga

SEQ ID NO: 116 - RNA, 23 nt, Mus musculus
uaaagugcuuauagugcagguag

SEQ ID NO: 117 - RNA, 22 nt, Mus musculus
uagcuuaucagacugauguuga

SEQ ID NO: 118 - RNA, 22 nt, Mus musculus
aagcugccaguugaagaacugu

SEQ ID NO: 119 - RNA, 21 nt, Mus musculus
aucacauugccagggauuucc

SEQ ID NO: 120 - RNA, 23 nt, Mus musculus
aucacauugccagggauuaccac

SEQ ID NO: 121 - RNA, 22 nt, Mus musculus
uggcucaguucagcaggaacag

SEQ ID NO: 122 - RNA, 22 nt, Mus musculus
uucaaguaauccaggauaggcu

SEQ ID NO: 123 - RNA, 22 nt, Mus musculus
uucaaguaauucaggauagguu

SEQ ID NO: 124 - RNA, 22 nt, Mus musculus
uucacaguggcuaaguuccgcu

SEQ ID NO: 125 - RNA, 20 nt, Mus musculus
uucacaguggcuaaguucug

SEQ ID NO: 126 - RNA, 22 nt, Mus musculus
cuagcaccaucugaaaucgguu

SEQ ID NO: 127 - RNA, 23 nt, Mus musculus
uagcaccauuugaaaucaguguu

SEQ ID NO: 128 - RNA, 22 nt, Mus musculus
uagcaccauuugaaaucgguua

SEQ ID NO: 129 - RNA, 23 nt, Mus musculus
uguaaacauccucgacuggaagc

SEQ ID NO: 130 - RNA, 22 nt, Mus musculus
cuuucagucggauguuugcagc

SEQ ID NO: 131 - RNA, 21 nt, Mus musculus
uguaaacauccuacacucagc

SEQ ID NO: 132 - RNA, 23 nt, Mus musculus
uguaaacauccuacacucucagc

SEQ ID NO: 133 - RNA, 22 nt, Mus musculus
uguaaacauccccgacuggaag

SEQ ID NO: 134 - RNA, 20 nt, Mus musculus
acccguagauccgaucuugu

SEQ ID NO: 135 - RNA, 22 nt, Mus musculus
cacccguagaaccgaccuugcg

SEQ ID NO: 136 - RNA, 20 nt, Mus musculus
uacaguacugugauaacuga

SEQ ID NO: 137 - RNA, 23 nt, Mus musculus
uggagugugacaaugguguuugu

SEQ ID NO: 138 - RNA, 23 nt, Mus musculus
uggagugugacaaugguguuuga

SEQ ID NO: 139 - RNA, 22 nt, Mus musculus
uuaaggcacgcggugaaugcca

SEQ ID NO: 140 - RNA, 21 nt, Mus musculus
uuaaggcacgcgggugaaugc

SEQ ID NO: 141 - RNA, 23 nt, Mus musculus
ucccugagacccuuuaaccugug

SEQ ID NO: 142 - RNA, 22 nt, Mus musculus
ucccugagacccuaacuuguga

SEQ ID NO: 143 - RNA, 21 nt, Mus musculus
ucguaccgugaguaauaaugc

SEQ ID NO: 144 - RNA, 21 nt, Mus musculus
cauuauuacuuuugguacgcg

SEQ ID NO: 145 - RNA, 22 nt, Mus musculus
ucggauccgucugagcuuggcu

SEQ ID NO: 146 - RNA, 22 nt, Mus musculus
ucacagugaaccggucucuuuu

SEQ ID NO: 147 - RNA, 21 nt, Mus musculus
cuuuuuucggucugggcuugc

SEQ ID NO: 148 - RNA, 22 nt, Mus musculus
cuuuuugcggucugggcuugcu

SEQ ID NO: 149 - RNA, 20 nt, Mus musculus
cagugcaauguuaaaagggc

SEQ ID NO: 150 - RNA, 23 nt, Mus musculus
uaacagucuacagccauggucgu

SEQ ID NO: 151 - RNA, 22 nt, Mus musculus
uugguccccuucaaccagcugu

SEQ ID NO: 152 - RNA, 22 nt, Mus musculus
ugugacugguugaccagaggga

SEQ ID NO: 153 - RNA, 24 nt, Mus musculus
uauggcuuuuuauuccuaugugaa

SEQ ID NO: 154 - RNA, 23 nt, Mus musculus
acuccauuuguuuugaugaugga

SEQ ID NO: 155 - RNA, 22 nt, Mus musculus
uauugcuuaagaauacgcguag

SEQ ID NO: 156 - RNA, 17 nt, Mus musculus
agcugguguugugaauc

SEQ ID NO: 157 - RNA, 18 nt, Mus musculus
ucuacagugcacgugucu

SEQ ID NO: 158 - RNA, 21 nt, Mus musculus
agugguuuuacccuaugguag

SEQ ID NO: 159 - RNA, 21 nt, Mus musculus
aacacugucugguaaagaugg

SEQ ID NO: 160 - RNA, 20 nt, Mus musculus
cauaaaguagaaagcacuac

SEQ ID NO: 161 - RNA, 22 nt, Mus musculus
uguaguguuuccuacuuuaugg

SEQ ID NO: 162 - RNA, 22 nt, Mus musculus
ugagaugaagcacuguagcuca

SEQ ID NO: 163 - RNA, 22 nt, Mus musculus
uacaguauagaugauguacuag

SEQ ID NO: 164 - RNA, 24 nt, Mus musculus
guccaguuuucccaggaaucccuu

SEQ ID NO: 165 - RNA, 23 nt, Mus musculus
ugagaacugaauuccauggguuu

SEQ ID NO: 166 - RNA, 21 nt, Mus musculus
guguguggaaaugcuucugcc

SEQ ID NO: 167 - RNA, 22 nt, Mus musculus
ucagugcacuacagaacuuugu

SEQ ID NO: 168 - RNA, 22 nt, Mus musculus
ucuggcuccgugucuucacucc

SEQ ID NO: 169 - RNA, 23 nt, Mus musculus
ucucccaacccuuguaccagugu

SEQ ID NO: 170 - RNA, 22 nt, Mus musculus
cuagacugaggcuccuugaggu

SEQ ID NO: 171 - RNA, 21 nt, Mus musculus
ucagugcaugacagaacuugg

SEQ ID NO: 172 - RNA, 20 nt, Mus musculus
uugcauagucacaaaaguga

SEQ ID NO: 173 - RNA, 22 nt, Mus musculus
uagguuauccguguugccuucg

SEQ ID NO: 174 - RNA, 22 nt, Mus musculus
uuaaugcuaauugugauagggg

SEQ ID NO: 175 - RNA, 23 nt, Mus musculus
aacauucaacgcugucggugagu

SEQ ID NO: 176 - RNA, 22 nt, Mus musculus
uuuggcaaugguagaacucaca

SEQ ID NO: 177 - RNA, 23 nt, Mus musculus
uauggcacugguagaauucacug

SEQ ID NO: 178 - RNA, 22 nt, Mus musculus
uggacggagaacugauaagggu

SEQ ID NO: 179 - RNA, 18 nt, Mus musculus
uggagagaaaggcaguuc

SEQ ID NO: 180 - RNA, 23 nt, Mus musculus
caaagaauucuccuuuugggcuu

SEQ ID NO: 181 - RNA, 22 nt, Mus musculus
ucgugucuuguguugcagccgg

SEQ ID NO: 182 - RNA, 22 nt, Mus musculus
caucccuugcaugguggagggu

SEQ ID NO: 183 - RNA, 23 nt, Mus musculus
gugccuacugagcugacaucagu

SEQ ID NO: 184 - RNA, 22 nt, Mus musculus
ugauauguuugauauauuaggu

SEQ ID NO: 185 - RNA, 22 nt, Mus musculus
caacggaaucccaaaagcagcu

SEQ ID NO: 186 - RNA, 18 nt, Mus musculus
cugaccuaugaauugaca

SEQ ID NO: 187 - RNA, 21 nt, Mus musculus
aacuggccuacaaagucccag

SEQ ID NO: 188 - RNA, 22 nt, Mus musculus
uguaacagcaacuccaugugga

SEQ ID NO: 189 - RNA, 21 nt, Mus musculus
uagcagcacagaaauauuggc

SEQ ID NO: 190 - RNA, 21 nt, Mus musculus
uagguaguuucauguuguugg

SEQ ID NO: 191 - RNA, 22 nt, Mus musculus
cccaguguucagacuaccuguu

SEQ ID NO: 192 - RNA, 22 nt, Mus musculus
uacaguagucugcacauugguu

SEQ ID NO: 193 - RNA, 22 nt, Mus musculus
uaacacugucugguaacgaugu

SEQ ID NO: 194 - RNA, 23 nt, Mus musculus
uaauacugccugguaaugaugac

SEQ ID NO: 195 - RNA, 21 nt, Mus musculus
uacucaguaaggcauuguucu

SEQ ID NO: 196 - RNA, 22 nt, Mus musculus
agagguauagcgcaugggaaga

SEQ ID NO: 197 - RNA, 23 nt, Mus musculus
gugaaauguuuaggaccacuaga

SEQ ID NO: 198 - RNA, 23 nt, Mus musculus
uucccuuugucauccuaugccug

SEQ ID NO: 199 - RNA, 22 nt, Mus musculus
uccuucauuccaccggagucug

SEQ ID NO: 200 - RNA, 22 nt, Mus musculus
uggaauguaaggaagugugugg

SEQ ID NO: 201 - RNA, 23 nt, Mus musculus
gcuucuccuggcucuccucccuc

SEQ ID NO: 202 - RNA, 22 nt, Mus musculus
auaagacgagcaaaaagcuugu

SEQ ID NO: 203 - RNA, 22 nt, Mus musculus
ugagguaguagguugugugguu

SEQ ID NO: 204 - RNA, 22 nt, Mus musculus
ugagguaguagguuguauaguu

SEQ ID NO: 205 - RNA, 22 nt, Mus musculus
ugagguaguagguuguaugguu

SEQ ID NO: 206 - RNA, 21 nt, Mus musculus
agagguaguagguugcauagu

SEQ ID NO: 207 - RNA, 21 nt, Mus musculus
ugagguaggagguuguauagu

SEQ ID NO: 208 - RNA, 22 nt, Mus musculus
ugagguaguagauuguauaguu

SEQ ID NO: 209 - RNA, 22 nt, Mus musculus
ugagguaguagauuguauaguu

SEQ ID NO: 210 - RNA, 22 nt, Mus musculus
ugagguaguaguuuguacagua

SEQ ID NO: 211 - RNA, 22 nt, Mus musculus
ugagguaguaguguguacaguu

SEQ ID NO: 212 - RNA, 19 nt, Mus musculus
ugagguaguaguuugugcu